(19) United States
(12) Patent Application Publication
(10) Pub. No.: US 2006/0127366 A1
(43) Pub. Date: Jun. 15, 2006

(54) Method and device for inducing biological processes by micro-organs

(76) Inventors: Eduardo N. Mitrani, Jerusalem (IL); Dov Eliezer Mitrani, Jerusalem (IL)

Correspondence Address: Martin Moynihan;Anthony Castorina, Suite 207, 2001 Jefferson Davis Highway, Arlington, VA, 22202, US

(21) Appl. No.: 10/519,838

(22) PCT Filed: Jul. 13, 2003
(86) PCT No.: PCT/IL03/00578

§ 371 Date: Dec. 8, 2005

Related U.S. Application Data
(63) Continuation-in-part of application No. 10/193,136, filed on Jul. 12, 2002, now abandoned.
Application No. 10/193,136 is a continuation-in-part of application No. 10/009,520, filed on May 3, 2002.
(60) Provisional application No. 60/140,748, filed on Jun. 25, 1999.

(30) Foreign Application Priority Data
May 19, 2003 (IT) 155985

(51) Int. Cl. A61K 35/12

(52) U.S. Cl. 424/93.7

(57) ABSTRACT

A method, extract, and pharmaceutical composition for inducing angiogenesis in a tissue of a mammal, and of a device for the preparation and delivery of micro-organs into a mammal, are provided.

### FIELD AND BACKGROUND OF THE INVENTION

The present invention relates to a method, extract and pharmaceutical composition for inducing angiogenesis in a tissue of a mammal, and to a device for the preparation and delivery of micro-organs (also refereed to herein as micro-organ explants), into a mammal.

During the last few years numerous research studies have provided new insights into the molecular mechanisms which induce and regulate cell growth, and in particular, angiogenesis. The discovery of angiogenic growth factors such as vascular endothelial growth factor (VEGF), basic fibroblast growth factor (bFGF), angiopoietin and others, has led researchers to consider the use of these factors as agents for revascularization of ischemic tissue regions. Several different approaches utilizing either gene therapy or recombinant protein technology have been attempted. Although preliminary results in animals were promising, clinical tests so far conducted, produced disappointing results (Ferrara and Alitalo, 1999 Nature Medicine 5(12): 1359-1364).

The lack of success at the clinical level can be attributed, at least in part, to the gene therapy or recombinant protein technology utilized in these experiments.

It has been shown that in vivo angiogenesis is effected and regulated by a complex and dynamic set of factors, including both stimulators and inhibitors (see Iruela-Arispe and Dvorak, 1997 Thrombosis and Haemostasis 78(1), 672-677, Gale and Yancopolous, 1999 Genes and Development 13, 1055-1066). In addition, it is thought that a long-term sustained stimulus is required to induce angiogenesis. Therefore, the current gene therapy and recombinant growth factors techniques, which do not address these issues, cannot produce the conditions necessary for promoting in vivo angiogenesis.

Recently, the inventor of the present invention have described a method for producing micro-organs which can be sustained outside the body in an autonomously functional state for extended periods of time. Such micro-organs, their preparation preservation and some uses thereof are described, for example, in U.S. Pat. No. 5,888,720; U.S. patent application Ser. No. 09/425,233, and in PCT/US98/00594, which are incorporated herein by reference.

### SUMMARY OF THE INVENTION

According to one aspect of the present invention, there is provided a method of inducing angiogenesis in a tissue of a first mammal, the method comprising the step of implanting at least one micro-organ within the tissue of the first mammal, said at least one micro-organ being for producing a plurality of angiogenic factors and thereby inducing angiogenesis.

According to an additional aspect of the present invention, said at least one micro-organ is derived from organ tissue of a second mammal.

According to an additional aspect of the present invention, the first mammal and said second mammal are a single individual mammal.

According to an additional aspect of the present invention, said organ is selected from the group consisting of a lung, a liver, a kidney, a muscle, a spleen a skin and a heart.

According to an additional aspect of the present invention, said at least one micro-organ includes two or more cell types.

According to an additional aspect of the present invention, the first mammal is a human being.

According to an additional aspect of the present invention, said at least one micro-organ is cultured outside the body for at least four hours prior to implantation within the tissue of the first mammal.

According to an additional aspect of the present invention, said at least one micro-organ is prepared so as to retain viability when implanted within the tissue of the first mammal.

According to an additional aspect of the present invention, said at least one micro-organ has dimensions, such that cells positioned deepest within said at least one micro-organ are at least about 80-100 microns and not more than about 225-375 microns away from a nearest surface of said at least one micro-organ.

According to an additional aspect of the present invention each of said plurality of angiogenic factors posses a unique pattern within said at least one micro-organ.

According to an additional aspect of the present invention, at least a portion of cells of said at least one micro-organ include at least one exogenous polynucleotide sequence selected for regulating angiogenesis.

According to an additional aspect of the present invention, said at least one exogenous polynucleotide sequence is integrated into a genome of said at least a portion of said cells of said at least one micro-organ.

According to an additional aspect of the present invention, said at least one exogenous polynucleotide sequence is designed for regulating expression of at least one angiogenic factor of said plurality of angiogenic factors.

According to an additional aspect of the present invention, said at least one exogenous polynucleotide sequence includes an enhancer or a suppresser sequence.

According to an additional aspect of the present invention, an expression product of said at least one exogenous polynucleotide sequence is capable of regulating the expression of at least one angiogenic factor of said plurality of angiogenic factors.

According to an additional aspect of the present invention, said at least one exogenous polynucleotide sequence encodes at least one recombinant angiogenic factor.

According to another aspect of the present invention, there is provided a method of inducing angiogenesis in a tissue of a first mammal, the method comprising:

(a) extracting soluble molecules from at least one micro-organ; and

(b) administering at least one predetermined dose of said soluble molecules extracted in step (a) into the tissue of the first mammal.

According to an additional aspect of the present invention, said soluble molecules are mixed with a pharmaceutically acceptable carrier prior to step (b).

According to an additional aspect of the present invention, said at least one micro-organ is derived from organ tissue of a second mammal;

According to an additional aspect of the present invention, said at least one micro-organ is cultured at least four hours prior to extraction of said soluble molecules.

According to additional aspect of the present invention, said at least one micro-organ has dimensions, such that cells positioned deepest within said at least one micro-organ are at least about 80-100 microns and not more than about 225-375 microns away from a nearest surface of said at least one micro-organ.

According to another aspect of the present invention, there is provided a pharmaceutical composition comprising, as an active ingredient, a soluble molecule extract from at least one micro-organ and a pharmaceutically acceptable carrier.

According to another aspect of the present invention, there is provided a micro-organ comprising a plurality of cells, wherein at least a portion of said plurality of said cells including at least one exogenous polynucleotide sequence, said at least one exogenous polynucleotide sequence being capable of regulating expression of at least one angiogenic factor expressed in said cells.

According to an additional aspect of the present invention, the micro-organ is derived from organ tissue of a second mammal.

According to an additional aspect of-the present invention, the first mammal and said second mammal are a single individual mammal.

According to an additional aspect of the present invention, said organ is selected from the group consisting of a lung, a liver, other gut derived organs, a kidney, a spleen and a heart.

According to an additional aspect of the present invention, said at least one micro-organ includes two or more cell types.

According to an additional aspect of the present invention, the micro-organ has dimensions, such that cells positioned deepest within the micro-organ are at least about 80-100 microns and not more than about 225-375 microns away from a nearest surface of the micro-organ.

According to an additional aspect of the present invention, said at least one exogenous polynucleotide sequence is integrated into a genome of said at least a portion of said plurality of said cells.

According to an additional aspect of the present invention, said at least one. exogenous polynucleotide sequence includes an enhancer or a suppressor sequence.

According to an additional aspect of the present invention, an expression portion of said at least one exogenous polynucleotide sequence is capable of regulating the expression of said at least one angiogenic factor.

According to another aspect of the present invention, there is provided a method of inducing angiogenesis in a tissue of a first mammal, the method comprising:

culturing at least one micro-organ in a growth medium to thereby generate a conditioned medium;

collecting said conditioned medium following at least one predetermined time period of culturing; and

administering at least one predetermined dose of said conditioned medium collected in step (b) into the tissue of the first mammal to thereby induce angiogenesis in the tissue.

According to an additional aspect of the present invention, said at least one micro-organ is derived from organ tissue of a second mammal.

According to an additional aspect of the present invention, said at least one micro-organ is cultured at least four hours prior to collection of said conditioned medium.

According to an additional aspect of the present invention, said at least one micro-organ has dimensions, such that cells positioned deepest within said at least one micro-organ are at least about 80-100 microns and not more than about 225-375 microns away from a nearest surface of said at least one micro-organ.

According to an additional aspect of the present invention, said growth medium is a minimal essential medium.

According to another aspect of the present invention there is provided apparatus for generating micro-organs from a tissue biopsy, and for implanting the micro-organs into a subject, the apparatus comprising:

(a) a cutting chamber for cutting the tissue biopsy into a plurality of micro-organs; and

(b) an implanting mechanism for implanting the plurality of micro-organs into the subject, said implanting mechanism being operably coupled to said cutting chamber.

According to an additional aspect of the present invention, said cutting chamber has an inlet/outlet for introducing and removing reagents.

According to an additional aspect of the present invention, said cutting chamber has an inlet for introducing the tissue biopsy therein.

According to an additional aspect of the present invention, said apparatus comprises a viability testing chamber operably coupled to said cutting chamber for testing a viability of at least one sacrificial micro-organ of said plurality of micro-organs.

According to an additional aspect of the present invention, said implanting mechanism comprises a multi-channel implanter and corresponding advancing elements for advancing said plurality of micro-organs from said cutting chamber to said multi-channel implanter and further for implanting the plurality of micro-organs into the subject. According to an additional aspect of the present invention, said apparatus comprises a processing chamber being operably coupled to said cutting chamber and said implanting mechanism for processing said micro-organs prior to said implanting.

According to an additional aspect of the present invention, said processing chamber has an inlet/outlet for introducing and removing processing reagents.

According to an additional aspect of the present invention, said cutting chamber comprises a cutting mechanism having a plurality of blades movable to cut the tissue biopsy into said plurality of micro-organs.

According to an additional aspect of the present invention, said blades are so disposed with respect to one another such that once the tissue biopsy is cut into said plurality of micro-organs, each of said micro-organs such that cells positioned deepest within a micro-organ of said plurality of micro-organs are at least about 80-100 microns and not more than 225-375 microns away from a nearest surface of said micro-organ.

According to an additional aspect of the present invention, said plurality of blades has a translatable angled cutting edge.

According to an alternative aspect of the present invention, each of said plurality of blades is a ratable disc-blade.

According to another aspect of the present invention there is provided apparatus for generating micro-organs from a tissue biopsy, the apparatus comprising:

(a) a cutting chamber for cutting the tissue biopsy into a plurality of micro-organs; and

(b) a viability testing chamber operably coupled to said cutting chamber for testing a viability of at least one sacrificial micro-organ of said plurality of micro-organs.

According to an additional aspect of the present invention, said cutting chamber has an inlet/outlet for introducing and removing reagents.

According to an additional aspect of the present invention, said cutting chamber has an inlet for introducing the tissue biopsy therein.

According to an additional aspect of the present invention, said cutting chamber comprises a cutting mechanism having a plurality of blades movable to cut the tissue biopsy into said plurality of micro-organs.

According to an additional aspect of the present invention, said blades are so disposed with respect to one another such that once the tissue biopsy is cut into said plurality of micro-organs, each of said micro-organs such that cells positioned deepest within a micro-organ of said plurality of micro-organs are at least about 80-100 microns and not more than 225-375 microns away from a nearest surface of said micro-organ.

According to an additional aspect of the present invention, each of said plurality of blades has a translatable angled cutting edge.

According to an alternative aspect of the present invention, each of said plurality of blades is a ratable disc-blade.

According to another aspect of the present invention there is provided apparatus for generating micro-organs from a tissue biopsy, the apparatus comprising:

(a) a cutting chamber for cutting the tissue biopsy into a plurality of micro-organs;

(b) a processing chamber being operably, coupled to said cutting chamber for processing said micro-organs;

(c) an advancing mechanism for advancing said micro-organs from said cutting chamber into said processing chamber.

According to an additional aspect of the present invention, said processing chamber has an inlet/outlet for introducing and removing processing reagents.

According to an additional aspect of the present invention, said cutting chamber has an inlet/outlet for introducing and removing reagents.

According to an additional aspect of the present invention, said cutting chamber has an inlet for introducing the tissue biopsy therein.

According to an additional aspect of the present invention, said cutting chamber comprises a cutting mechanism having a plurality of blades movable to cut the tissue biopsy into said plurality of micro-organs.

According to an additional aspect of the present invention, said blades are so disposed with respect to one another such that once the tissue biopsy is cut into said plurality of micro-organs, each of said micro-organs such that cells positioned deepest within a micro-organ of said plurality of micro-organs are at least about 80-100 microns and not more than 225-375 microns away from a nearest surface of said micro-organ.

According to an additional aspect of the present invention, each of said plurality of blades has a translatable angled cutting edge.

According to an alternative aspect of the present invention, each of said plurality of blades is a ratable disc-blade.

According to another aspect of the present invention there is provided a method of generating micro-organs from a tissue biopsy and for implanting the micro-organs into a subject, the method comprising:

providing an apparatus which comprises:

(a) a cutting chamber for cutting the tissue biopsy into a plurality of micro-organs; and

(b) an implanting mechanism for implanting the plurality of micro-organs into the subject, said implanting mechanism being operably coupled to said cutting chamber.

placing the tissue biopsy is said cutting chamber and cutting the tissue biopsy into the plurality of micro-organs; and

using said implanting mechanism for implanting the plurality of micro-organs into the subject.

According to an additional aspect of the, present invention, the micro-organs, serve as angiopumps.

According to an additional aspect of the present invention, said cutting chamber has an inlet/outlet for introducing and removing reagents, the method further comprising washing said micro-organs in said cutting chamber prior to using said implanting mechanism for implanting the plurality of micro-organs into the subject.

According to an additional aspect of the present invention, said cutting chamber has an inlet for introducing the tissue biopsy therein, the method comprising placing the tissue biopsy in said cutting chamber through said inlet.

According to an additional aspect of the present invention, said apparatus further comprises a viability testing chamber operably coupled to said cutting chamber for testing a viability of at least one sacrificial micro-organ of said plurality of micro-organs, the method further comprising testing said viability of said at least one sacrificial micro-organ of said plurality of micro-organs prior to using said implanting mechanism for implanting the plurality of micro-organs into the subject.

According to an additional aspect of the present invention, said implanting mechanism comprises a multi-channel implanter and corresponding advancing elements for advancing said plurality of micro-organs from said cutting chamber to said multi-channel implanter and further for implanting the plurality of micro-organs into the subject, the method comprising implanting the plurality of micro-organs into the subject using said advancing elements.

According to an additional aspect of the present invention, said apparatus comprises a processing chamber being operably coupled to said cutting chamber and said implanting mechanism for processing said micro-organs prior to said implanting, the method further comprising processing said micro-organs prior to said implanting.

According to an additional aspect of the present invention, said processing said micro-organs prior to said implanting comprises at least one a process selected from the group consisting of washing, transforming, culturing, and a combination thereof.

According to an additional aspect of the present invention, said processing said micro-organs prior to said implanting comprises culturing for at least one hour.

According to an additional aspect of the present invention, said processing said micro-organs prior to said implanting comprises transforming by introducing to at least a portion of cells of said micro-organs at least one exogenous polynucleotide sequence selected for regulating angiogenesis.

According to an additional aspect of the present invention, said at least one exogenous polynucleotide sequence is integrated into a genome of said at least said portion of said cells of said micro-organs.

According to an additional aspect of the present invention, said at least one exogenous polynucleotide sequence is designed for regulating expression of at least one angiogenic factor of said plurality of angiogenic factors.

According to an additional aspect of the present invention, said at least one exogenous polynucleotide sequence includes an enhancer or a suppresser sequence.

According to an additional aspect of the present invention, said at least one exogenous polynucleotide sequence is capable of regulating the expression of at least one angiogenic factor of said plurality of angiogenic factors.

According to an additional aspect of the present invention, said at least one exogenous polynucleotide sequence encodes at least one recombinant angiogenic factor.

According to an additional aspect of the present invention, said processing chamber has an inlet/outlet for introducing and removing processing reagents, the method comprising introducing at least one processing reagent into said processing chamber through said inlet/outlet.

According to an additional aspect of the present invention, said cutting chamber comprises a cutting mechanism having a plurality of blades movable to cut the tissue biopsy into said plurality of micro-organs, the method comprising using said plurality of blades to cut the tissue biopsy into, said plurality of micro-organs.

According to an additional aspect of the present invention, said cutting chamber is designed and constructed such that once the tissue biopsy is cut into said plurality of micro-organs, each of said micro-organs such that cells positioned deepest within a micro-organ of said plurality of micro-organs are at least about 80-100 microns and not more than 225-375 microns away from a nearest surface of said micro-organ, the method further comprising using said cutting chamber to cut the tissue biopsy into said plurality of micro-organs each of said micro-organs such that cells positioned deepest within a micro-organ of said plurality of micro-organs are at least about 80-100 microns and not more than 225-375 microns away from said nearest surface of said micro-organ.

According to an additional aspect of the present invention, each of said plurality of blades has a translatable angled cutting edge, the method comprising translating said angled cutting edge with respect to the tissue biopsy, so as to cut the tissue biopsy into said plurality of micro-organs.

According to an alternative aspect of the present invention, each of said plurality of blades is a rotable disc-blade, the method comprising moving said rotable disc-blade with respect to the tissue biopsy, so as to cut the tissue biopsy into said plurality of micro-organs.

According to an additional aspect of the present invention, said tissue biopsy is derived from a tissue or organ selected from the group consisting of lung, liver, kidney, muscle, spleen, skin, heart, lymph node and bone marrow.

According to an additional aspect of the present invention, a donor of the tissue biopsy and the subject are the same individual.

According to an alternative aspect of the present invention, a donor of the tissue biopsy and the subject are different individuals.

According to an additional aspect of the present invention, a donor of the tissue biopsy is a human.

According to an alternative aspect of the present invention, a donor of the tissue biopsy is a non-human mammal.

According to an additional aspect of the present invention, said subject is a non-human mammal.

According to an alternative aspect of the present invention, said subject is a human.

According to an additional aspect of the present invention, said implanting the plurality of micro-organs into the subject is effected via transmucosal or parenteral administration routes.

According to an additional aspect of the present invention, said transmucosal or parenteral administration routes are selected from the group consisting of intramuscular, subcutaneous, intramedullary intrathecal, direct intraventricular, intravenous, intraperitoneal, intranasal and intraocular administration routes.

According to a preferred aspect of the present invention, there is provided a device for micro-organ preparation and delivery, comprising:

a tissue scraper, for obtaining a tissue biopsy;

a tissue cutter, for cutting the tissue biopsy into a plurality of fragments, forming a plurality of micro-organs: and

at least one implanting device, detachably coupled to said tissue cutter, for receiving a micro-organ, of said plurality of micro-organs, when coupled to said tissue cutter, and for implanting said micro-organ into a subject, after decoupling from said tissue cutter.

According to an additional aspect of the present invention, said device is sealed within a base, a ramp, and a casing.

According to an additional aspect of the present invention, said device includes a control system.

According to an additional aspect of the present invention, said device includes at least one automated travel mechanism for transferring the tissue biopsy from one region of said device to another.

According to an additional aspect of the present invention, said tissue scraper is adapted for scraping said tissue to a predetermined width.

According to an additional aspect of the present invention, said tissue scraper is adapted for scraping said tissue to a predetermined length.

According to an additional aspect of the present invention, said tissue scraper is adapted for scraping said tissue to a predetermined thickness.

According to an additional aspect of the present invention, said tissue scraper has a replaceable blade.

According to an additional aspect of the present invention, said device includes a washing apparatus for rinsing the tissue biopsy.

According to an additional aspect of the present invention, said washing apparatus is operative for applying a medium to the tissue biopsy.

According to an additional aspect of the present invention, said device is further operative as a tissue treatment chamber.

According to an additional aspect of the present invention said device includes apparatus for controlling the temperature therein.

According to an additional aspect of the present invention, said tissue cutter comprises a plurality of parallel, surgical-grade blades, designed to cut the tissue biopsy into said plurality of fragments, forming said micro-organs, such that cells positioned deepest within any one of said micro-organs are at least about 80-100 microns and not more than about 225-375 microns away from a nearest surface.

According to an additional aspect of the present invention, said tissue cutter comprises a plurality of parallel surgical-grade blades, arranged at an angle to the tissue biopsy.

According to an alternative aspect of the present invention, said tissue cutter comprises a plurality of parallel surgical-grade blades, arranged as rotable disc-blades.

According to an additional aspect of the present invention, said device comprises a viability testing chamber for testing a viability of at least one micro-organ of said plurality of micro-organs.

According to an additional aspect of-the present invention, said tissue cutter is operative to cut the tissue biopsy, to form said micro-organs, and to arrange each of said micro-organs on a single micro-organ guide of a plurality of micro-organ guides, in a single operation.

According to an additional aspect of the present invention, said at least one implanting device includes a slim housing, adapted for percutaneous insertion, and operable to receive one of said plurality of micro-organ guides.

According to an additional aspect of the present invention, said at least one implanting device includes a plurality of implanting devices, each operable to receive one of said plurality of micro-organ guides.

According to an additional aspect of the present invention, each of said plurality of micro-organ guides includes a position marker for indicating when said micro-organ, arranged on it, is positioned for implanting.

According to an additional aspect of the present invention, each of said micro-organ guides includes a notch for breaking off a distal portion thereof, to allow said micro-organ, arranged on it, to form a leading edge.

According to an additional aspect of the present invention each of said plurality of micro-organ guides includes a position marker for indicating micro-organ, arranged on it, is implanted.

According to an additional aspect of the present invention, said device is disposable.

According to another preferred aspect of the present invention, there is provided a method for micro-organ preparation and delivery, comprising:

scraping a tissue biopsy;

cutting the tissue biopsy to a plurality of fragments, forming a plurality of micro-organs; and

implanting at least one of said plurality of one micro-organs.

According to another preferred aspect of the present invention, there is provided a method for micro-organ preparation and delivery, comprising:

employing a device for micro-organ preparation and delivery, which includes:

a tissue scraper, for obtaining a tissue biopsy;

a tissue cutter, for cutting the tissue biopsy into a plurality of fragments, forming a plurality of micro-organs: and at least one implanting device, detachably coupled to said tissue cutter, for receiving a micro-organ, of said plurality of micro-organs, when coupled to said tissue utter, and for implanting said micro-organ into a subject, after decoupling from said issue cutter;

scraping the tissue biopsy, with said tissue scraper;

cutting the tissue biopsy to said plurality of fragments, forming said plurality of micro-organs, with said tissue cutter;

mounting said micro-organ, of said plurality of micro-organs, on said at least one implanting device;

decoupling said at least one implanting device; and

implanting said micro-organ with said at least one implanting device.

According to an additional aspect of the present invention, the micro-organ serves as an angiopump.

According to an additional aspect of the present invention, said method includes treating the tissue biopsy prior to implanting.

According to an additional aspect of the present invention, said treating is from the group consisting of washing, transforming, culturing, and a combination thereof.

According to an additional aspect of the present invention, said cutting includes arranging each of said micro-organs on a single micro-organ guide of a plurality of micro-organ guides.

According to an additional aspect of the present invention, said method includes disposing said device after a single use

According to an additional aspect of the present invention, said cutting further includes cutting to a first plurality of tissue fragments, forming a first plurality of micro-organs; and

said implanting further includes implanting a second plurality of micro-organs, wherein said second plurality is selected from the group consisting of a plurality which is equal to said first plurality, a plurality which is smaller than said second plurality by one, and a plurality which is smaller than said second plurality by two.

According to an alternative aspect of the present invention, said cutting further includes cutting to a first plurality of tissue fragments, forming a first plurality of micro-organs; and

said implanting further includes implanting a second plurality of fragments, wherein said second plurality is smaller than said first plurality by one, wherein said method further includes using an edge fragment for a viability test.

According to an alternative aspect of the present invention, said cutting further includes cutting to a first plurality of tissue fragments, forming a first plurality of micro-organs; and

said implanting further includes implanting a second plurality of tissue fragments, wherein said second plurality is smaller than said first plurality by two;

wherein said method further includes using a first edge fragment for a viability test; and

discarding a second edge fragment.

According to an additional aspect of the present invention, said cutting includes cutting the tissue biopsy into said plurality of fragments, forming said micro-organs such that cells positioned deepest within any of said micro-organs are at least about 80 microns and not more than about 375 microns away from a nearest surface.

According to an additional aspect of the present invention, said cutting includes cutting the tissue biopsy into said plurality of fragments, forming said micro-organs, such that cells positioned deepest within any one of said micro-organs are at least about 100 microns and not more than about 225 microns away from a nearest surface.

According to an additional aspect of the present invention, said implanting further includes implanting a plurality of micro-organs within a preselected area of said subject, for a predetermined area concentration of micro-organs.

According to an additional aspect of the present invention, said implanting further includes implanting a plurality of micro-organs within a preselected volume of said subject, for a predetermined volume concentration of micro-organs.

According to an additional aspect of the present invention, said tissue biopsy is a split-thickness tissue biopsy.

The present invention successfully addresses the shortcomings of the presently known configurations by providing a method, extract, and pharmaceutical composition for inducing angiogenesis in a tissue of a mammal, and a device for the preparation and delivery of micro-organs into a mammal.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a photograph showing neo-vascularization around an implanted micro-organ (marked with arrow),

FIG. 2 is a graph illustrating the relative levels of various angiogenic factors expressed in transplanted micro-organs. Ang1—angiopoietin 1, Ang2—angiopoietin 2, MEF2C —myocyte enhancer factor 2C, VEGF—vascular endothelial growth factor;

FIG. 3 is an angiogenic factor-specific RT-PCR of RNA extracted from micro-organs cultured outside the body for various time points following preparation. Actin—beta-actin (control);

FIG. 4 is a graph representing semi-quantitative data obtained by densitometry of the RT-PCR products shown in FIG. 3, normalized to the intensity of the beta-actin RT-PCR product (control);

FIG. 5 is a histogram representing the gating pattern of common iliac-ligated rats implanted with micro-organs or sham implanted (control). (n)=13. P values for the three time groups (from left to right) are 0.16, 1 and 0.841. Scores: 0-full functionality 9-total inability to move the limb, 10 loss of the limb;

FIG. 6 is a histogram representing the same experimental group as in FIG. 5 with the exception that the animals were now exerted prior to scoring gating behavior. P values for the three time groups are (from left to right) 0.0001, 0.0069 and 0.06;

FIG. 7 is a histogram representing the gating pattern of common iliac-ligated mice implanted with micro-organs or sham implanted. Scores: 0-full functionality 9-total inability to move the limb, 10 loss of the limb. P values for the three time groups are (from left to right) 0.00025, 0.00571 and 0.07362;

FIG. 8 is an image illustrating a mouse spleen derived micro-organ (marked with MC arrow) six months following implantation into a subcutaneous region of a syngeneic mouse. One of the newly formed blood vessels surrounding the micro-organ is marked with an arrow;

FIG. 9 is an image illustrating a rat cornea implanted with lung micro-organs from a syngeneic rat. The implanted micro-organ (marked with arrow) is surrounded by newly formed blood vessels;

FIGS. 10A-B schematically illustrate a device for micro-organ preparation and delivery, in accordance with a preferred embodiment of the present invention;

FIG. 11 schematically illustrates a tissue scraper in accordance with a preferred embodiment of the present invention;

FIG. 12 schematically illustrates the tissue scraper, in accordance with a preferred embodiment of the present invention;

FIGS. 13A-B schematically illustrate a tissue cutter, in accordance with a preferred embodiment of the present invention;

FIGS. 14A-D schematically illustrate the tissue cutter, in accordance with a preferred embodiment of the present invention;

FIG. 15 schematically illustrates the tissue cutter, when cutting is complete, in accordance with a preferred embodiment of the present invention;

FIGS. 16A-B schematically illustrate applying a medium for keeping micro-organs moist, in accordance with a preferred embodiment of the present invention;

FIGS. 17A-E, schematically illustrate the steps in inserting micro-organs into implanting devices, in accordance with a preferred embodiment of the present invention;

FIGS. 18A-C schematically illustrate the steps in implanting the micro-organs in a body, in accordance with a preferred embodiment of the present invention;

FIGS. 19A-C illustrate angiogenesis in implanted skin micro-organs (SMOs) 1, 3 and 7 days following implantation (arrows indicate newly formed blood vessels);

FIGS. 20A-B illustrate Regional blood flow in implanted SMOs (FIG. 20A) as compared to flow induced by lung MO (FIG. 20B). Fluorescent beads were used to determine the flow intensity;

FIGS. 21A-B illustrate vessel formation in young vs. old SMOs one month following implantation in young mice;

FIGS. 22A-B illustrate blood flow in young vs. old SMOs two weeks following implantation in young mice;

FIGS. 23A-G are photographs taken under a fluorescent microscope illustrating vessel formation in muscle tissue devoid of implanted SMOs (FIGS. 23A, G) and SMO implanted muscle tissue (FIGS. 23B-D);

FIG. 24 illustrates blood vessel formation in a single SMO rescued seven days following implantation in the recipient rabbit;

FIGS. 25A-N schematically illustrate stages in the tooling and operation of micro-forceps according to the present invention;

FIG. 26 is a schematic illustration of a second device for micro-organ preparation and delivery, in accordance with a preferred embodiment of the present invention;

FIGS. 27A-D schematically illustrate the second device with the cutting stages by plurality of blades, in accordance with a preferred embodiment of the present invention; and

FIGS. 28A-C, 29A-C schematically illustrate the use of the micro-forceps of the invention in context of the method and device of the present invention.

### DESCRIPTION OF THE PREFERRED EMBODIMENTS

The present invention is of a method, extract, and pharmaceutical composition for inducing angiogenesis in a tissue of a mammal, and a device for the preparation and delivery of micro-organs into a mammal.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or exemplified in the examples section that follows. The invention is capable of other embodiments or of being practiced or carried out in various ways. Also, it is to be understood that the phraseology and terminology employed herein is for the purpose of description and should not be regarded as limiting.

As used herein, the term “micro-organ” refers to organ tissue which is removed from a body and which is prepared, as is further described below, in a manner conducive for cell viability and function. Such preparation may include culturing outside the body for a predetermined time period. The term “angiopump” refers to micro-organs processed, preferably verified for cell viability and prepared in a manner ready, but not necessarily utilized, for immediate administration.

Complex multicellular organisms rely on a vascular network to support the needs of each and every cell for oxygen, nutrients and waste removal. This complex network of blood vessels is created and sustained through the process of angiogenesis. In humans, the deterioration of the vascular network leads to occlusive arterial disease, which is the leading cause for morbidity and mortality in the Western world. Most currently available therapeutic options are based on surgical or other invasive procedures, such as vascular bypass or angioplasty. These solutions are for the most part successful but may be short lived or not applicable to all patients. Since angiogenesis is a fundamental component of tissue and organ genesis, most tissues retain the capacity to induce new vessel formation during regeneration. Thus, the inventors of the present invention postulate that tissue which is removed from the body is in essence at least attempting to undergo regeneration and thus can be utilized as an angiogenic stimulant, or more broadly for stimulation of cell growth processes.

The present invention provides a new approach to induce angiogenesis and other cell growth properties, which approach is based on the use of micro-organs. Such. micro-organs retain the basic micro-architecture of the tissues of origin while at the same time are prepared such that cells of an organ explant are not more than 100-450 micron away from a source of nutrients and gases. Such micro-organs function autonomously and remain viable for extended period of time both as ex-vivo cultures and in the implanted state. Furthermore such micro-organs not only function but secrete a whole repertoire of angiogenic factors which induce a significant vascular network in their vicinity.

It will be appreciated that although micro-organs can be utilized -immediately following preparation, in some cases culturing outside the body for extended periods of-time may be advantageous in order to increase viability. For example, in cases where soluble molecules are to be extracted, culturing of micro-organs is performed for predetermined time periods, which can be as short as 4 hours or as long as days or weeks.

Thus, the use of these micro-organs or extracts derived therefrom for inducing angiogenesis and other cell growth properties is dependent on the preservation of cellular function for various periods of time, prior to implantation. The present invention is based, in part, upon the discovery that under defined circumstances, growth of cells in different tissue layers of an organ explant, e.g., mesenchymal and epithelial layers, can be activated to proliferate, differentiate and function in culture.

The cell-cell and cell-matrix interactions provided in the explant itself are sufficient to support cellular homeostasis, thereby sustaining the microarchitecture and function of the organ for prolonged periods of time. As used herein, the term “homeostasis” is defined as equilibrium between cell proliferation and cell loss.

The support of cellular homeostasis preserves, for example, the natural cell-cell and cell-matrix interactions occurring in the source organ. Thus, orientation of the cells with respect to each other or to another anchorage substrate, as well as the presence or absence of regulatory substances such as hormones, permits the appropriate maintenance of biochemical and biological activity of the source organ. Moreover, the micro-organ can be maintained in culture without significant necrosis for at least 48 days.

Source of Explants for the Micro-Organ:

Examples of mammals from which the micro-organs can be isolated include humans and other primates, swine, such as wholly or partially inbred swine (e.g., miniature swine, and transgenic swine), rodents, etc. Examples of suitable organs include, but are not limited to, liver, lung, other gut derived organs, heart, spleen, kidney, skin and pancreas.

The Growth Media:

There are a large number of tissue culture media that exist for culturing cells from animals. Some of these are complex and some are simple. While it is expected that micro-organs may grow in complex media, it has been shown in U.S. patent application Ser. No. 08/482,364 that cultures can be maintained in a simple medium such as Dulbecco's Minimal Essential Media (DMEM). Furthermore, although the micro-organs may be cultured in a media containing sera or other biological extracts such as pituitary extract, it has been shown in U.S. patent application Ser. No. 08/482,364 that neither sera nor any other biological extract is required. Moreover, the micro-organ cultures can be maintained in the absence of sera for extended periods of time. In preferred embodiments of the invention, growth factors are not included in the media during maintenance of the micro-organ cultures in vitro.

The point regarding growth in minimal media is important. At the present, most media or systems for prolonged growth of mammalian cells incorporate undefined proteins or use feeder cells to provide proteins necessary to sustain such growth. Because the presence of such undefined proteins can interfere with the intended end use of the micro-organs, it will generally be desirable to culture the explants under conditions to minimize the presence of undefined proteins.

As used herein the language “minimal medium” refers to a chemically defined medium, which includes only the nutrients that are required by the cells to survive and proliferate in culture. Typically, minimal medium is free of biological extracts, e.g., growth factors, serum, pituitary extract, or other substances, which are not necessary to support the survival and proliferation of a cell population in culture. For example, minimal medium generally includes at least one amino acid, at least one vitamin, at least one salt, at least one antibiotic, at least one indicator, e.g., phenol red, used to determine hydrogen ion concentration, glucose, and at least one antibiotic, and other miscellaneous components necessary for the survival and proliferation of the cells. Minimal medium is serum-free. A variety of minimal media are commercially available from Gibco BRL, Gaithersburg, Md., as minimal essential media.

However, while growth factors and regulatory factors need not be added to the media, the addition of such factors, or the inoculation of other specialized cells may be used to enhance, alter or modulate proliferation and cell maturation in the cultures. The growth and activity of cells in culture can be affected by a variety of growth factors such as insulin, growth hormone, somatomedins, colony stimulating factors, erythropoietin, epidermal growth factor, hepatic erythropoietic factor (hepatopoietin), and other cell growth factors such as prostaglandins, interleukins, and naturally-occurring negative growth factors,. fibroblast growth factors, and members of the transforming growth factor-beta family.

Culture Vessel:

The micro-organs may be maintained in any suitable culture vessel and may be maintained at 37° C. in 5% CO₂. The cultures may be shaken for improved aeration.

With respect to the culture vessel in/on, which the micro-organs are preferably provided, it is noted that in a preferred embodiment such a vessel may generally be of any material and/or shape. A number of different materials may be used to form the vessel, including but not limited to nylon (polyamides), dacron (polyesters), polystyrene, polypropylene, polyacrylates, polyvinyl compounds (e.g., polyvinylchloride), polycarbonate (PVC), polytetrafluorethylene (PTFE; teflon), thermanox (TPX), nitrocellulose, cotton, polyglycolic acid (PGA), cat gut sutures, cellulose, gelatin, dextran, etc. Any of these materials may be woven into a mesh.

Where the cultures are to be maintained for long periods of time or cryopreserved, non-degradable materials such as nylon, dacron, polystyrene, polycarbonate, polyacrylates, polyvinyls, teflons, cotton or the like may be preferred. A convenient nylon mesh which could be used in accordance with the invention is Nitex, a nylon filtration mesh having an average pore size of 210 μm and an average nylon fiber diameter of 90 μm (Tetko, Inc., N.Y.).

Dimensions of the Explant:

In addition to isolating an explant which retains the cell-cell, cell-matrix and cell-stroma architecture of the originating tissue, the dimensions of the explant are crucial to the viability of the cells therein, e.g., where the micro-organ is intended to be sustained for prolonged periods of time, such as 7-21 days or longer.

Accordingly, the dimensions of the tissue or organ are selected to provide diffusion of adequate nutrients and gases such as oxygen to every cell in the three dimensional micro-organ, as well as diffusion of cellular waste out of the explant so as to minimize cellular toxicity and concomitant death due to localization of the waste in the micro-organ. Accordingly, the size of the explant is determined by the requirement for a minimum level of accessibility to each cell in the absence of specialized delivery structures or synthetic substrates. It has been discovered, as described in U.S. patent application Ser. No. 08/482,364 that this accessibility can be maintained if the surface to volume index falls within a certain range.

This selected range of surface area to volume index provides the cells access to nutrients and to avenues of waste disposal by diffusion in a manner similar to cells in a monolayer. This level of accessibility can be attained and maintained if the surface area to volume index, defined herein, as “Aleph or Aleph index” is at least about 2.6 mm⁻¹. The third dimension has been ignored in determining the surface area to volume index because variation in the third dimension causes ratiometric variation in both volume and surface area. However, when determining Aleph, a and x should be defined as the two smallest dimensions of the tissue fragment.

As used herein, “Aleph” refers to a surface area to volume index given by a formula 1/x+1/a, wherein x=tissue thickness and a=width of tissue in mm. in preferred embodiments, the Aleph of an explant is in the range of from about 2.7 mm⁻¹ to about 25 mm⁻¹, more preferably in the range of from about 2.7 mm⁻¹ to about 15 mm⁻¹, and even more preferably in the range of from about 2.7 mm⁻¹ to about 10 mm⁻¹.

Examples of Aleph are provided in Table 1 wherein, for example, a tissue having a thickness (x) of 0.1 mm and a width (a) of 1 mm would have an Aleph index of 11 mm⁻¹.

TABLE 1
Different values for the surface area to volume ratio index “Aleph”, as a
function of a (width) and x (thickness) in mm⁻¹

| | Values of Aleph | | | | |
|---|---|---|---|---|---|
| x (mm) | a = 1 | a = 2 | a = 3 | a = 4 | a = 5 |
| 0.1 | 11 | 10.51 | 10.33 | 10.2 | 10.2 |
| 0.2 | 6 | 5.5 | 5.33 | 5.25 | 5.2 |
| 0.3 | 4.3 | 3.83 | 3.67 | 3.58 | 3.53 |
| 0.4 | 3.5 | 3 | 2.83 | 2.75 | 2.7 |
| 0.5 | 3 | 2.5 | 2.33 | 2.25 | 2.2 |
| 0.6 | 2.66 | 2.16 | 2 | 1.91 | 1.87 |
| 0.7 | 2.4 | 1.92 | 1.76 | 1.68 | 1.63 |
| 0.8 | 2.25 | 1.75 | 1.58 | 1.5 | 1.45 |
| 0.9 | 2.11 | 1.61 | 1.44 | 1.36 | 1.31 |
| 1.0 | 2 | 1.5 | 1.33 | 1.25 | 1.2 |
| 1.2 | 1.83 | 1.3 | 1.16 | 1.08 | 1.03 |
| 1.3 | 1.77 | 1.26 | 1.1 | 1.02 | 0.96 |
| 1.6 | 1.625 | 1.13 | 0.96 | 0.88 | 0.83 |
| 2.0 | 1.5 | 1 | 0.83 | 0.75 | 0.7 |

Thus, for example, cells positioned deepest within an individual micro-organ are at least 80 microns, and not more than 375 microns, away from a nearest surface of the individual micro-organ. These measurements facilitate the preservation of in vivo architecture, while concurrently ensuring that no cell is farther than 225-300 microns from a source of gases and nutrients.

Without being bound by any particular theory, a number of factors provided by the three-dimensional culture system may contribute to its success.

First, the appropriate choice of the explant size, e.g., by use of the above Aleph calculations, provides appropriate surface area to volume ratio for adequate diffusion of nutrients to all cells of the explant, and adequate diffusion of cellular waste away from all cells in the explant.

Second, because of the three-dimensionality of the explant, various cells continue to actively grow, in contrast to cells in monolayer cultures, which grow to confluence, exhibit contact inhibition, and cease to grow and divide. The elaboration of growth and regulatory factors by replicating cells of the explant may be partially responsible for stimulating proliferation and regulating differentiation of cells in culture, e.g., even for the micro-organ, which is static in terms of overall volume.

Third, the three-dimensional matrix of the explant retains a spatial distribution of cellular elements, which closely approximates that found in the counterpart organ in vivo.

Fourth, the cell-cell and cell-matrix interactions may allow the establishment of localized microenvironments conducive to cellular maturation. It has been recognized that maintenance of a differentiated cellular phenotype requires not only growth/differentiation factors but also the appropriate cellular interactions.

While reducing the present invention to practice, and as is further described in the Examples section hereinbelow, it was discovered that when micro-organs are implanted in a recipient, they provide a sustained dosage of a complex repertoire of angiogenic and other growth factors and cytokines thus leading to the formation of new blood vessels in the implanted tissues of the host. It was also discovered that micro-organs could reverse ischemia in host tissues in both normal and aging animals. In addition, it was also revealed that micro-organs cultured in vitro also express the same repertoire of angiogenic and other growth factors and cytokines.

Thus, according to one aspect of the present invention there is provided a method of inducing angiogenesis and cell growth in a tissue of a mammal, such as, for example a human being. The method is effected by implanting at least one micro-organ within the tissue of the mammal. Examples of tissue suitable for micro-organ implantation include but are not limited to, organ tissue or muscle tissue.

Such implantation can be effected via standard surgical techniques or via implanting of micro-organ preparations into the intended tissue regions of the mammal utilizing specially adapted syringes employing a needle of a gauge suitable for the administration of micro-organs.

The micro-organs utilized for implantation are preferably prepared from an organ tissue of the implanted mammal or a syngeneic mammal, although xenogeneic tissue can also be utilized for the preparation of the micro-organs providing measures are taken prior to, or during implantation, so as to avoid graft rejection and/or graft versus host disease (GVHD). Numerous methods for preventing or alleviating graft rejection or GVHD are known in the art and as such no further detail is given herein.

It will be appreciated that to facilitate transplantation of the explants which may be subject to immunological attack by the host, e.g., where xenogenic grafting is used, such as swine-human transplantations, the micro-organ can be inserted into or encapsulated by rechargeable or biodegradable devices and then transplanted into the recipient subject. Gene products produced by such cells can then be delivered via, for example, polymeric devices designed for the controlled delivery compounds, e.g., drugs, including proteinaceous biopharmaceuticals. A variety of biocompatible polymers (including hydrogels), including both biodegradable and non-degradable polymers, can be used to form an implant for the sustained release of a gene product of the cell populations of the invention at a particular target site. The generation of such implants is generally known in the art. See, for example, Concise Encyclopedia of Medical & Dental Materials, ed. By David Williams: (MIT Press: Cambridge, Mass. 1990) the Sabel et al. U.S. Pat. No. 4,883,666; Aebischer et al. U.S. Pat. No. 4,892,538; Aebischer et al. U.S. Pat. No. 5,106,627; Lim U.S. Pat. No. 4,391,909; and Sefton U.S. Pat. No. 4,353,888.

According to one preferred embodiment of the present invention, at least a portion of cells of the micro-organ includes at least one exogenous polynucleotide sequence. Such polynucleotide sequence(s) are preferably stably integrated into the genome of these cells although transient polynucleotide sequences can also be utilized. It will be appreciated that such exogenous polynucleotides can be introduced into the cells of the micro-organ following explantation from the organ tissue of the mammal or alternatively the mammal can be transformed with the exogenous polynucleotides prior to preparation of organ tissue or organs. Methods for transforming mammalian cells are described in detail hereinbelow.

Such exogenous polynucleotide(s) can serve for enhancing angiogenesis or cell growth by, for example, up-regulating or down-regulating the expression of one or more endogenous angiogenic or growth factors or cytokines expressed within these cells. In this case, the polynucleotide(s) can include trans-, or cis-acting enhancer or suppresser elements which regulate either the transcription or translation of the endogenous angiogenic and/or growth factors or cytokines expressed within these cells. Numerous examples of suitable translational or transcriptional regulatory elements, which can be utilized in mammalian cells, are known in the art.

For example, transcriptional regulatory elements are cis or trans acting elements, which are necessary for activation of transcription from specific promoters (Carey et al. (1989), J. Mol. Biol., 209:423-432; Cress et al. (1991) Science, 251:87-90; and Sadowski et al. (1988), Nature, 335:563-564).

Translational activators are exemplified by the cauliflower miosaic virus translational activator (TAV). See, for example Futterer and Hohn (1991) EMBO J. 10:3887-3896. In this system a di-cistronic mRNA is produced. That is, two coding regions are transcribed in the same mRNA from the same promoter. In the absence of TAV, only the first cistron is translated by the ribosomes. However, in cells expressing TAV, both cistions are translated.

The polynucleotide sequence of cis acting regulatory elements can be introduced into cells of micro-organs via commonly practiced gene knock-in techniques. For a review of gene knock-in/out methodology see, for example, U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270, 1991; Capecchi, Science 244:1288-1292, 1989; Davies et al., Nucleic Acids Research, 20 (11) 2693-2698, 1992; Dickinson et al., Human Molecular Genetics, 2(8):1299-1302, 1993; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991; Jakobovits et al., Nature, 362:255-261 1993; Lamb et al., Nature Genetics, 5: 22-29, 1993; Pearson and Choi, Proc. Natl. Acad. Sci. USA, 1993, 90:10578-82; Rothstein, Methods in Enzymology, 194:281-301, 1991; Schedl et al., Nature, 362: 258-261, 1993; Strauss et al., Science, 259:1904-1907, 1993, WO 94/23049, WO93/14200, WO 94/06908 and WO 94/28123 also provide information.

Down-regulation of endogenous angiogenic and/or growth factors or cytokines can also be achieved via antisense RNA. In this case the exogenous polynucleotide(s) can encode sequences which are complementary to the mRNA sequences of the angiogenic and/or growth factors or cytokines transcribed in the cells of the micro-organ. Down regulation can also be effected via gene knock-out techniques.

Up-regulation can also be achieved by overexpressing or by providing a high copy number of one or more angiogenic and/or growth factor or cytokine coding sequences. In this case, the exogenous polynucleotide sequences can encode one or more angiogenic or growth factors or cytokines such as but not limited to VEGF, bFGF, Ang1 or Ang2 which can be placed under the transcriptional control of a suitable promoter of a mammalian expression vector. Suitable mammalian expression vectors include, but are not limited to, pcDNA3, pcDNA3.1 (+/−), pZeoSV2(+/−), pSecTag2, pDisplay, pEF/myc/cyto, pCMV/myc/cyto, pCR3.1, and their derivatives, which are available from Invitrogen, pCI which is available from Promega, pBK-RSV and pBK-CMV which are available from Stratagene, pTRES which is available from Clontech.

Numerous methods are known in the art for introducing exogenous polynucleotide sequences into mammalian cells. Such methods include, but are not limited to, direct DNA uptake techniques, and virus or liposome mediated transformation (for further detail see, for example, “Methods in Enzymology” Vol. 1-317, Academic Press). Micro-organ bombardment with nucleic acid coated particles is also envisaged.

It will be appreciated that the angiogenic and/or growth factors or cytokines expressed in micro-organs can be extracted therefrom as a crude or refined extract in a soluble phase and utilized directly, or as part of a pharmaceutical composition for local administration into host tissues, e.g., in order to induce angiogenesis or other cell growth processes. It will further be appreciated that since micro-organs express different levels of the -various angiogenic and/or growth factors and cytokines at different time points following implantation or during culturing, one can extract soluble molecules from different micro-organ cultures at different time points, which when locally administered in a series, mimic the temporal expression of an implanted or cultured micro-organ.

Thus, according to another aspect of the present invention, there is provided another method of inducing angiogenesis or other cell processes in a tissue of a mammal. This method is effected by extracting soluble molecules from micro-organs and locally administering at least one predetermined dose of the soluble molecules extracted into the tissue of the mammal. Numerous methods of administering are known in the art. Detailed description of some of these methods is given hereinbelow with regards to pharmaceutical compositions.

As mentioned above and according to another preferred embodiment of the present invention the soluble extracts are included in a pharmaceutical composition which also includes a pharmaceutically acceptable carrier which serves for stabilizing and/or enhancing the accessibility or targeting of the soluble extract to target body tissues.

Examples of a pharmaceutically acceptable carrier include but are not limited to, a physiological solution, a viral capsid carrier, a liposome carrier, a micelle carrier, a complex cationic reagent carrier, a polycathion carrier such as poly-lysine and a cellular carrier.

The soluble extract, which constitutes the “active ingredient” of the pharmaceutical composition, can be administered to the individual via various administration modes.

Suitable routes of administration may, for example, include transmucosal or parenteral delivery, including intramuscular, subcutaneous and intramedullary implanting as well as intrathecal, direct intraventricular, intravenous, intraperitoneal, intranasal, and/or intraocular implanting.

Preferably, the composition or extract is administered in a local rather than a systemic manner, for example, via implanting directly into an ischemic tissue region of the individual.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For implanting, the active ingredient may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

The composition described herein may be formulated for parenteral administration, e.g., by bolus implanting or continuous infusion. Formulations for implanting may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active ingredient in water-soluble form. Additionally, suspensions of the active ingredients may be prepared as appropriate oily or water based implanting suspensions. Suitable lipophilic solvents or vehicles include fatty oils such as sesame oil, or synthetic fatty acids esters such as ethyl oleate, triglycerides or liposomes. Aqueous implanting suspensions may contain substances, which increase the viscosity of the suspension, such as sodium carboxymethyl cellulose, sorbitol or dextran. Optionally, the suspension may also contain suitable stabilizers or agents who increase the solubility of the active ingredients to allow for the preparation of highly concentrated solutions.

In addition, the composition of the present invention may be delivered via localized pumps, or time release reservoirs which can be implanted within ischemic tissues of the individual.

Since angiogenic and other growth factors and cytokines are typically secreted from producing cells, micro-organs can also be cultured in suitable media and the conditioned media which includes the secreted angiogenic factors can be collected at predetermined time points and utilized as described hereinabove with respect to the soluble extract.

Thus, according to yet another aspect of the present invention there is provided a method of inducing angiogenesis or other cell processes in a tissue of a first mammal. The method according to this aspect of the present invention is effected by culturing at least one micro-organ in a growth medium to thereby generate a conditioned medium, collecting the conditioned medium following at least one predetermined time period of culturing and administering at least one predetermined dose of the conditioned medium into the tissue of the first mammal to thereby induce angiogenesis or other cell growth processes in the tissue.

Preferably, the growth medium is a minimal essential medium (described hereinabove) which does not contain undefined proteins or other growth factors which may interfere with the intended function of the conditioned media or which may cause undesired reactions in the administered mammal.

It will be appreciated that the collected conditioned media can be processed using chromatographic techniques, such as affinity columns and the like, so as to yield a substantially pure preparations which include an array of angiogenic or other growth factors suitable for inducing angiogenesis or other cell growth processes when administered to a mammal.

It will further be appreciated that the conditioned medium and the soluble extract described herein can also be derived from micro-organs which include exogenous polynucleotides as described hereinabove. In such cases, if the exogenous polynucleotides utilized encode angiogenic or other growth factors or cytokines, the sequence of such exogenous polynucleotides is selected suitable for the intended administered mammal. For example, in cases where the soluble extract or conditioned medium is administered to human recipients, human or humanized exogenous polynucleotides are preferably utilized.

The micro-organs according to the teachings of the present invention can be utilized following preparation, or alternatively they can be cryopreserved and stored at −160° C. until use. For example, micro-organs can be cryopreserved by gradual freezing in the presence of 10% DMSO (Dimethyl Sulfoxide) and 20% serum.

This can be effected, for example, by encapsulating the micro-organs within planar sheets, (e.g., a semi-permeable matrix such as alginate) and inserting these encapsulated micro-organs into a sealable sterile synthetic plastic bag of dimensions closely similar to that of the encapsulated micro-organs. The bag would contain one plastic tubing input at one end and one plastic tubing output at the opposite end of the bag. The sealed plastic bag containing the planar sheet with the micro-organs could then be perfused with standard culture medium such as Ham's F12 with 10% DMSO and 20% serum and gradually frozen and stored at −160° C.

An important goal in cardiovascular medicine would be to replace surgical bypasses with therapeutic angiogenesis. Yet, in spite of the considerable efficacy observed when angiogenic factors were used in animal models of coronary or limb ischemia, the clinical results have been disappointing. Recently, it has been suggested that clinical failure may be due to the application of the angiogenic factor or the combination of factors utilized. The angiogenesis method of the present invention overcomes such limitations of prior art methods.

The present invention brings forth a novel approach, which recognizes that angiogenesis and other cell growth processes are complex, highly regulated and sustained processes, mediated by several regulatory factors. The results presented by the present invention provide a model, which allows studying the induction of angiogenesis, and cell growth both in and out of the body, and, as such, allows for the establishment of a pattern of expression of key regulatory factors. The results presented herein show that implanted micro-organs express several key angiogenic and other cell growth factors in a coordinated manner, both in and out of the body. Furthermore, as shown by in vivo experiments, micro-organs function as genuine angiopumps not only by transcribing angiogenic and other growth factors, but also by inducing the formation of new blood vessels. Furthermore, the magnitude of the induction is such that the vessels formed are sufficient to irrigate the surrounding area and rescue artificially induced hypoxic tissue regions in mice and rats.

The model for ischemia in rats presented hereinbelow in the example section appears to mimic chronic ischemia since no irreversible damage has occurred. In untreated animals, the ischemia was apparent only after exertion. Presumably, there is enough collateral circulation to keep the limb viable but not enough to allow normal function when faced with an additional challenge. The implantation of micro-organs appears to have reversed this condition by increasing blood supply to ischemic regions. The results show a significant difference between the micro-organ-treated and the control groups which difference is undoubtedly due to the induction of angiogenesis and other cell growth processes by the micro-organs.

In the series of mouse in vivo rescue experiments presented herein the ischemic insult was increased. Mice have inferior collateral circulation to the hindlimbs due to less developed tail arteries as compared to rats. In this group, signs of acute irreversible ischemic damage such as gangrene and auto-amputation, were detected in the control group. This finding suggests that the present invention may also be useful for salvage procedures, though further testing is warranted.

In an additional series of trials presented below the ischemic challenge was further increased by inducing ischemia in previously diseased animals. Again, irreversible ischemic damage occurred only in the control animals. The damage to the control animals was so severe that stress tests were deemed superlative. Though the sample size was small the differences were marked. These results are particularyl important sinc they illustrate that micro-organs are capable of inducing angiogenesis and other cell growth processes even in tissues affected by some types of peripheral vascular-disease.

Thus the present invention provides methods and compositions for inducing and maintaining blood vessel formation and other cellular processes within host tissues for the purposes of stimulating cell growth, rescuing ischemic tissues and/or generating natural bypasses around blocked blood vessels.

The present invention provides methods and compositions for the development and production of viable, sterile angiopumps that can be administered quickly and easily in an outpatient setting. It will be appreciated that the procurement, testing and administration of the angiopumps can thus be accomplished most easily, or alternatively, can be similarly stored for administration at a later stage.

A novel device for the preparation and delivery of micro-organs is further provided and disclosed by the present invention. A detailed disclosure of the device is provided under Example 7 of the Examples section that follows.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example; “Molecular Cloning: A laboratory Manual” Sambrook et al, (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John. :Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes. I-III. Coligan J. E., ed. (1994); Stites et al. (eds), “Basic and Clinical Immunology” (8^{th} Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Micro Organs

Materials and Experimental Methods

Approval for animal experiments was obtained from the Animal Care and Use Committee of the Faculty of Science of the Hebrew University.

Micro-Organ Preparation:

Adult animals (C57B1/6 mice or Sprague Dawley rats) were sacrificed by asphyxiation with CO₂ and the lungs were removed under sterile conditions. The lungs were kept on ice and rinsed once with Ringer solution or DMEM including 4.5 gm/l D-glucose micro-organs were prepared by chopping the lungs with a Sorvall tissue chopper into pieces approximately 300 μm in width. micro-organs were rinsed twice wih DMEM containing 500 units/ml Penicillin, 0.5 mg/ml Streptomycin and 2 mM L-Glutamine (Biological Industries) and kept on ice until use.

Micro-Organ Implantation:

Adult C57BV6 mice were anesthetized using 0.6 mg Sodium Pentobarbitol per gram body weight. The mice were shaved, and an incision about 2 cm long was made in the skin at an area above the stomach. A hemostat was used to create subcutaneous “pockets” on both sides of the incision, and 8-9 micro-organs were implanted in each pocket; implantation was done by simply layering the micro-organs over the muscle layer. The incision was sutured and the animals were kept in a warm, lit room for several hours following which they were transferred to the animal house. Four animals were sacrificed at a time interval of either 4 hours, 24 hours, 72 hours or 7 days following implantation and the implanted micro-organs were dissected from surrounding tissues under a surgical microscope and utilized for RNA extraction. The extracted RNA was reverse transcribed and the resulting cDNA was used as a template for PCR analysis using standard methodology. The oligonucleotide primer sequences utilized in the PCR reaction, the expected product size and references are given in Table 2 .

TABLE 2

RT-PCR primer sequence and source

| | | | |
|---|---|---|---|
| Name | Genebank # | Sequence | Product |

| | | | |
|---|---|---|---|
| Ang2 | AF004326.1 | F: 5′- | 400 bp |
| | | CGTGGGTGGAGGAGGGTGGAC- | |
| | | 3′ | |
| | | (SEQ ID NO: 1) | |
| | | R: 5′- | |
| | | TGCGTCAAACCACCAGCCTCC- | |
| | | 3′ | |
| | | (SEQ ID NO: 2) | |
| β- | | F: 5′- | 310 |
| Actin | | TACCACAGGCATTGTGATGG- | bp**** |
| | | 3′ | |
| | | (SEQ ID NO: 3) | |
| | | R: 5′- | |
| | | AATAGTGATGACCTGGCCGT- | |
| | | 3′ | |
| | | (SEQ ID NO: 4) | |
| Ang1 | U83509 | F: 5′- | 273 |
| | | GGTCACACAG GGACAGCAGG- | bp*/** |
| | | 3′ | |
| | | (SEQ ID NO: 5) | |
| | | R: 5′- | |
| | | CCAAGGGCCGGATCAGCATGG- | |
| | | 3′ | |
| | | (SEQ ID NO: 6) | |
| VEGF | U41383 | F: 5′- | 444, |
| | | ACTTTCTGCTCTCTTGGGT-3′ | 573***/** |
| | | (SEQ ID NO: 7) | |
| | | R: 5′- | |
| | | CCGCCTTGGCTTGTCACA-3′ | |
| | | (SEQ ID NO: 8) | |

*Another discrete band, is oflen detected at approximately 320 bp - the origin of this band is unknown.
**Primers (Ang1) or primer sequence (VEGF) was kindly supplied by professor Eli Keshet, Israel.
***VEGF mRNA undergoes alternative splicing. PCR product sizes are 444 bp for VEGF₁₂₁, 573 bp for VEGF₁₆₅, and 645 bp for VEGF₁₈₉.
****Ibrahim et al. 1998 Biochimica et Biophysica Acta 1403, 254-264.

Densitometric Analysis and Quantification:

A 10 μl aliquot of each PCR reaction was electrophoresed in a 1.5% agarose gel stained with ethidium bromide. Gels were imaged utilizing a Macintosh Centris 660 AV computer and a Fujifilm Thermal Imaging System with a Toyo Optics TV zoom lens (75-125 mm, F=1.8, with a Colkin orange 02 filter). Densitometric analysis was performed using the public domain NIH 1.61 analysis software. Quantitation was done by normalizing the expression level of each PCR product to those obtained for β-Actin. All PCR reactions were performed in duplicate. Statistical analysis of the relative expression levels of the various VEGF isoforms was performed by comparison of the means, using Welsh's t-test on nonpaired samples before and after implantation.

Ischemic Tissue Rescue Experiments:

32 Sprague Dawley rats aged 1-4 months and weighing 200-300 grams were utilized. The left common Iliac of each rat was ligated and excised from rats anesthetized using 0.9-1.1 mg/gram body weight of Pentothal at the aortic bifurcation just proximal to the Iliac bifurcation. Sixteen rats were implanted wit h 3-4 micro-organs each 24 hours following the induction of ischemia. The micro-organs were implanted intramuscularly and subcutaneously along the Femoral artery (medially) and along the sciatic nerve (laterally). The remaining sixteen rats underwent sham implantation 24 hours following the induction of ischemia.

Twenty six C57B1/6 mice aged 1-3 months and weighing 19 to 27 grams were also tested. The left Common Iliac artery of anesthetized mice was ligated and excised at the aortic bifurcation just proximal to the Iliac bifurcation. 3-4 micro-organs were implanted in each mouse at 24 hours following the induction of ischemia. Nine mice were implanted intramuscularly and subcutaneoutsly along the Femoral artery (medially) and along the sciatic nerve (laterally) in the proximal left hindlimb. Seventeen control mice were prepared for implantation following ischemia induction but no implantation was performed. Animals that had venous or nervous damage during the operation as well as those that suffered from significant bleeding were excluded from the trial.

Seven old C57B1/6 mice aged 22 months and weighing 24 to 28 grams also underwent ligation and excision of the left Common Iliac artery as described above. Three were immediately implanted with micro-organs derived from normal healthy syngeneic mice. Four had immediate sham implantation. None suffered from venous or nervous damage or had significant bleeding during the operation.

Functional Assay:

The animals were tested on the first and second days following implantation to rule out nerve damage. The test consisted of swimming in a lukewarm water bath, which was set at a water level such that the animal needed to constantly exert all four limbs in order to stay afloat. The time limits for exercise were gradually increased. During the first week the time limit was 3 minutes or until efforts to remain afloat ceased. During the second week the limit was raised to 5 minutes, while from the third week onwards the time limit was 6 minutes. A scale from 0 to 10 was created to assess the degree of claudication. A score of 0-1 indicated normal or near normal gait. A score of 2-3 meant slight to moderate claudication with normal weight bearing. A score of 4-5 indicated moderate claudication with disturbance in weight bearing. A score of 6-7 indicated severe claudication.

A score of 8-9 indicated a non functioning limb, atrophy or contracture and a score of 10 meant gangrene or autoamputation. The scores were assigned by an independent observer not involved in the experiment and having no knowledge of previous animal treatments.

Angiography:

Angiography was performed on several rats at days 4, 14, 26 and 31 following implantation. The rats were anesthetized as previously described and a P10 catheter was introduced through the right superficial femoral artery and placed in the aorta. A bolus implanting of 1 cc Telebrix was injected and the animal was photographed every 0.5 seconds. Animals undergoing angiography were subsequently excluded from the trial groups.

Experimental Results

Implanted Micro-Organs Induce Angiogenesis:

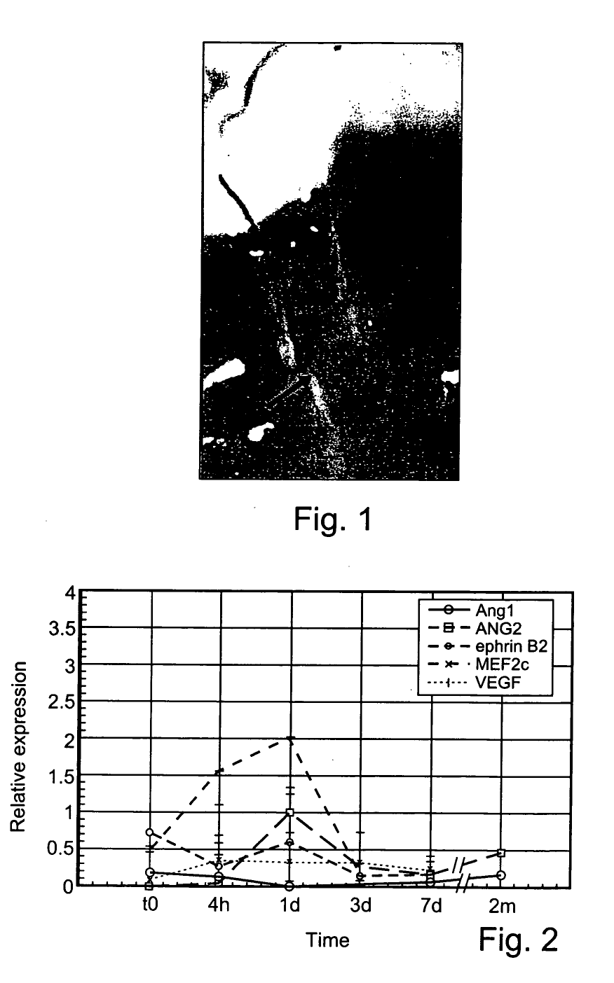

FIG. 1 illustrates the response of surrounding tissues to implanted micro-organs. When a micro-organ is implanted subcutaneously into a syngeneic animal, it induces an angiogenic response towards the micro-organ (arrow, FIG. 1). A major blood vessel forms and branches into smaller vessels, which branch into a net of capillaries, which surround the implanted micro-organ.

micro-organs transcribe a sustained and dynamic array of angiogenic growth factors when implanted subcutaneously into syngeneic mice. FIG. 2 illustrate a representative semi-quantitative analysis of several known angiogenic growth factors as determined from the RT-PCR analysis performed on RNA extracted from the micro-organs. As seen from the results, a strong induction of angiogenic factor expression occurs at 4 hours post implantation (PI). Following this initial induction, each individual growth factor follows a different expression pattern as is further detailed-below.

VEGF: VEGF transcription level continued to rise at 24 hours PI. At three days PI, transcription levels of VEGF decreased. In the days following, lower mRNA levels of this angiogenic factor were detected, which levels were probably necessary in order to maintain the neo-angiogenic state thus formed. At seven days PI, VEGF mRNA returned to a level similar to that detected in micro-organs at the time of implantation (to).

Angiopoietin 1: The level of Ang1 mRNA increased for the first 4 hours PI, although variation was high. At one to three days PI, transcription dropped to levels which are even lower than that detected for micro-organs at the time of implantation (to) (see Maisonpierre et al., 1997, Science 277, 55-60, Gale and Yancopolous, 1999 Ibid.). At seven days PI, Ang1 mRNA returned to a level similar to that detected at t₀.

Angioprotein 2: Ang2, the antagonist of Ang1, was transcribed at high levels at 24 hours P1. mRNA levels dropped at 3 and 7 days PI, although these levels were still higher than the levels detected at to, possibly due to ongoing vascular remodeling in and around the implanted micro-organ.

Thus, as is evident from these results, implanted micro-organs transcribe a dynamic array of factors, both stimulators and inhibitors which participate in the regulation of angiogenesis. This transcription pattern which is responsible for the generation of new blood vessels around the micro-organs is sustained over a period of at least one week PI.

Micro-organs transcribe a sustained and dynamic array of angiogenic growth factors when cultured:

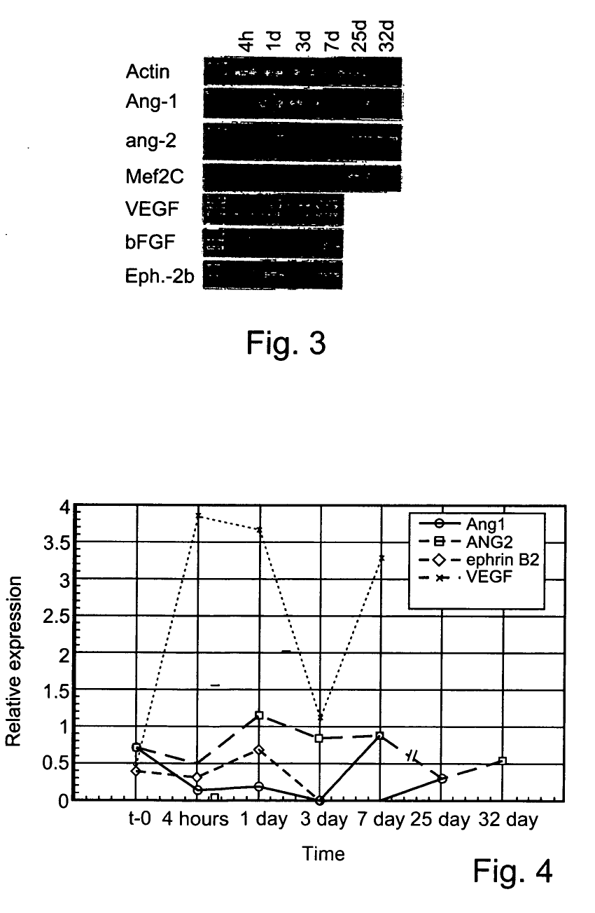

In order to determine the capacity of micro-organs to transcribe angiogenic factors when cultured ex-vivo, micro-organs prepared as described above, were grown in the absence of serum for periods of over one month. Samples were removed at various time points and assayed for the mRNA levels of the several factors. FIG. 4 illustrate a representative semi-quantitative analysis of several known angiogenic growth factors as determined from RT-PCR performed on RNA extracted from cultured micro-organs (FIG. 3). As shown in both Figures a strong induction of angiogenic factor expression occurs 4 hours following culturing. Following this initial induction, each different growth factor follows a different expression pattern as is described in detail below.

VEGF: VEGF expression levels continued to rise 24 hours after culturing. 3 days after culturing, the expression level of VEGF decreased only to increase again at 7 days PI. In the following days expression levels drop and VEGF expression returns to a level comparable to that expressed by micro-organs at the time of culturing.

Angiopoietin 1: The level of Ang1 expression increased for the first 4 hours following culturing although variation Was high. Expression dropped 1 to 3 days after culturing-to levels even lower than that detected at time of culturing. At seven days after culturing Ang1 expression returned to a level comparable to the level at time of culturing.

Angiopoietin 2: Ag2 the antagonist of Ang1, was expressed at a high level during the first day-after culturing. The expression levels were lower 3 and 7 days after culturing, although they are still higher than the expression level at time of culturing.

As is evident from these results, micro-organs which are cultured outside the body remain viable and functional for over a month in vitro and express a dynamic array of angiogenic factors, including both stimulators and inhibitors, which participate in the regulation of angiogenesis.

Implantation of micro-organs reverse ischemia in limbs of rats and mice:

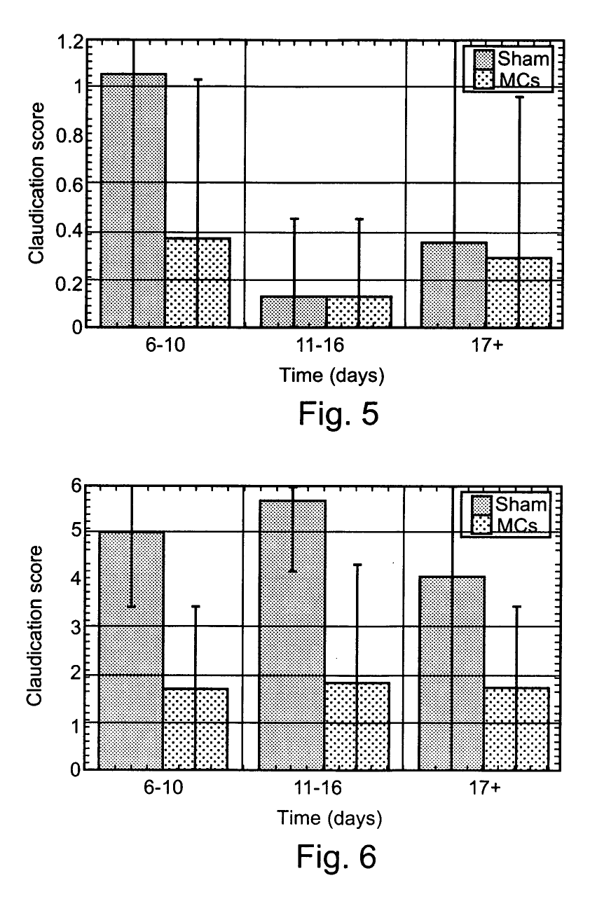

Series 1: The left common iliac artery of thirty two rats was ligated and excised as described above micro-organs implantation was conducted in sixteen of these rats while the sixteen remaining rats served as the control group (sham operations). All 32 rats survived the operation. No significant difference was detected between the two groups prior to exertion (FIG. 5). Following exertion, a significant difference was detected; the cumulated average claudication score for the control group was 4.8 whereas in the micro-organ implanted group the score was 1.6 (FIG. 6). Similar results were recorded throughout the study period. The control group scored 5 on days 6-10 post operation (PO), 5 at 11-15 days PO and 4 at day 17 PO. The scores for the micro-organ implanted group were 1.67, 1.5 and 1.7, respectively. It should be noted that the micro-organ implanted group included one rat with an average score of 6.5. A histological examination revealed necrotic micro-organ implants in this rat.

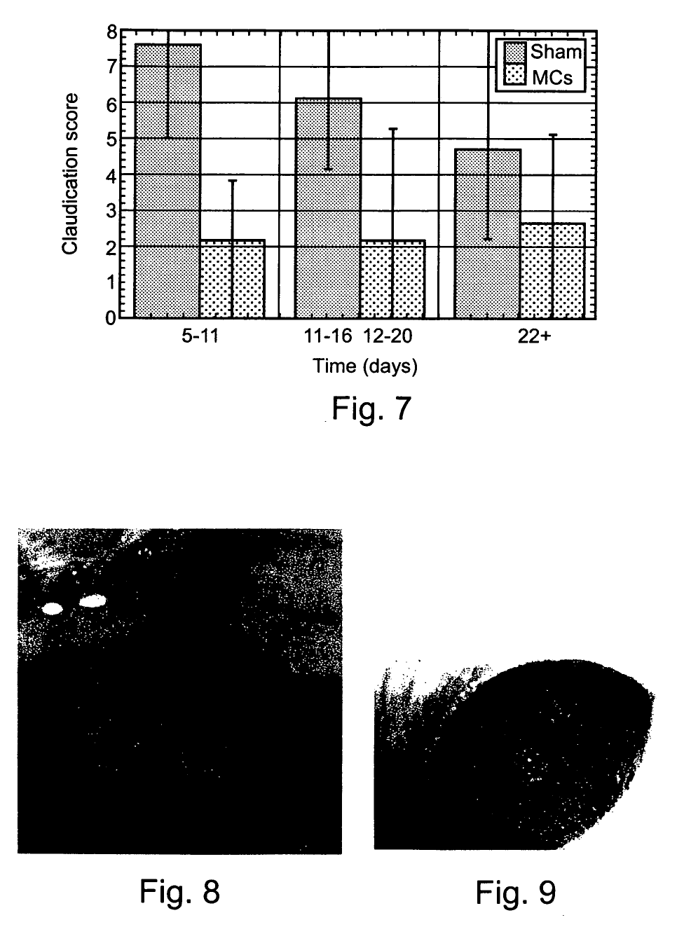

Series 2: Twenty six young C57B1/6 mice were operated as described above without operative damage or preoperative mortality micro-organ implantation was conducted in nine of these mice while seventeen served as control (sham operations). Of the 17 control mice, 4 developed gangrene on the ischemic-induced limb and died 2-3 days PO (23.5%). Another mouse from this group had autoamputation of an atrophied limb 8 days after operation (5.9%). None of the micro-organ implanted mice developed gangrene, autoamputation or postoperative death (0%). The average cumulated post exertion claudication score for the control group was 6 with scores of 7.7 on days 5-9 PO, 6.2 on days 13-19 PO and 4.1 on days 21-25 PO. The average cumulated claudication score for the micro-organ implanted group was 2.4, with scores of 1.8 on days 5-9 PO, 2.2 on days 13-19 PO and 3.1 on-days 21-25 PO (FIG. 7).

Micro-Organ Implantation Rescues Ischemic Limbs In Old Mice:

Series 3: Seven aged C57B1/6 mice were operated upon with no operative damage or death. Three mice received micro-organ implants and 4 served as control. Of the control group, 1 developed gangrene and died 3 days PO (25%) and one had autoamputation of an atrophied limb 5 days PO (25&). The remaining two mice had non functioning limbs at rest (a score of 8 on the claudication index). None of the micro-organ implanted mice developed gangrene or autoamputation (0%) and their average claudication score at one week was 5.7.

Implanted Micro-Organs are Viable, and Vascularized:

In sampled rat specimens the micro-organ implants were viable, with preserved architecture and no evidence of rejection. The micro-organs and surrounding muscle tissue was vascularized via macroscopically visible blood vessels.

Angiography reveals angiogenic activity in micro-organ-implanted rats:

Angiography was performed on days 4, 14, 26 and 31 PO. There were subtle but detectable differences between the micro-organ-treated groups and the control groups. Evidence of increased angiogenic activity in the implanted limb was detected as early as day 4 PO. New, medium sized blood vessels were visible in the implanted limb sixteen days PO.

### Example 2

### Spleen Micro-Organs

Mouse Spleen micro-organs were prepared from as described hereinabove and implanted into syngeneic mice. FIG. 8 illustrates a micro-organ (arrow) which was implanted subcutaneously into the syngeneic mouse and examined at six months following implantation. As is clearly demonstrated in FIG. 8, the micro-organ induced angiogenesis. In fact, the pattern of blood vessels formed gives the impression that-the micro-organ is micro-organ Was an inherent organ of the host.

### Example 3

### Cornea Implantation of Micro-Organs

The cornea is the only tissue of the body, which is devoid of blood vessels. As such, the cornea is an excellent model tissue for studying angiogenesis. Rat lung micro-organs were implanted in the corneas of syngeneic rats. As shown in FIG. 9, a most remarkable angiogenic pattern was also induced in the cornea. These remarkable results again verify that micro-organs are effective in inducing and promoting angiogenesis.

### Example 4

### Mouse Skin MOs Implanted in C57BL Mice

Materials and Experimental Methods:

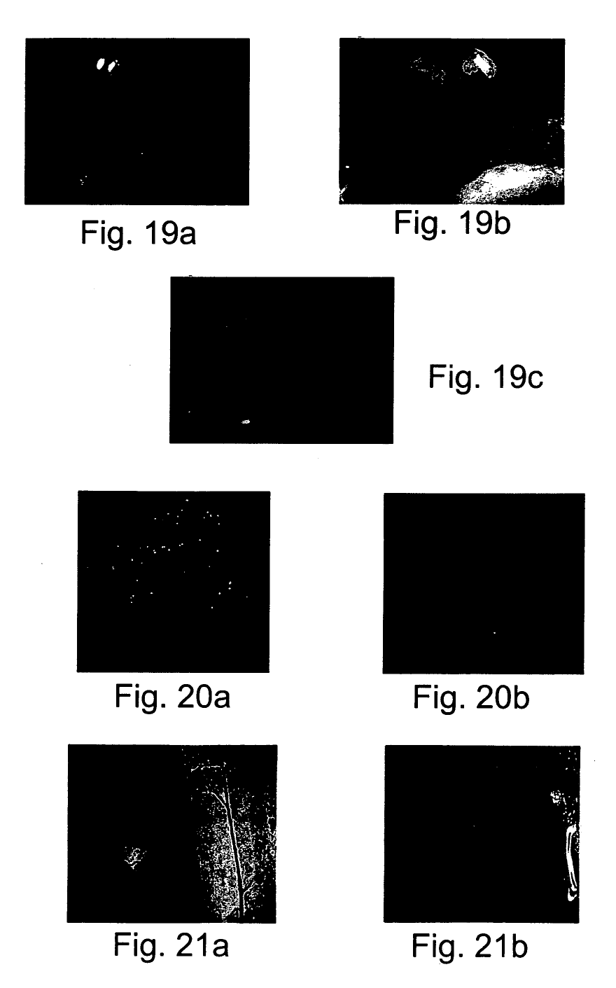

Adult C57BL mice were anesthetized using Sodium Pentobarbital. The mice were shaved, and an incision about 1 cm long was made in the skin at the center of the stomach. A haemostat was used to create subcutaneous pockets on both sides of the incision, and about 10 skin MOs (SMOs), prepared as described earlier, were placed side by side (on their side thus exposing all tissue layers) in each pocket. The incision was closed using surgical sutures. One, three, seven and thirty days following implantation, the recipient mice were sacrificed and the SMOs were excised from surrounding tissues under a surgical microscope (FIGS. 19A-C). Ten skin MOs were taken at time zero.

Determination of Regional Blood Flow:

SMO implanted Mice were anesthetized using Sodium Pentobarbital (0.06 mg per gram body weight) and the right carotid artery was cannulated using heparinized-saline (20 U/ml) filled PE-10 tubing which was narrowed at the portion inserted into the vessel. The tubing was utilized to inject 10⁵ polystyrene yellow-green fluorescent microspheres (Molecular probes 15 μm in diameter) into the left ventricle and 0.15 ml saline which was slowly injected into the left ventricle over a period of 30 seconds following injection of the microspheres.

The microspheres were found distributed throughout the implanted skin micro-organ indicating that blood was flowing into the SMO and that the vascular network had further expanded throughout the whole tissue (FIG. 20A).

### Example 5

### An Aging Model

It is a well known fact that as individuals age the risk of cardio and peripheral vascular diseases such as atherosclerotic increases while regenerative capabilities responsible for wound healing among other processes decrease.

One factor which may contribute to this increase in risk and decrease in regenerative capabilities is a decrease in the body's capacity to stimulate angiogenesis.

To verify this theory, SMOs from old mice were compared to SMOs from young mice as far as their capacity to stimulate angiogenesis in a recipient host.

Materials and Experimental Methods:

RNA extraction and cDNA synthesis: Total RNA was extracted from equal amount of skin MOs using the acid-guanidine-phenol method described by Chomczynski, P. (1994) in Cell biology: a laboratory handbook, ed. E, C. J. (Academic press, Vol. 1, pp. 680-683 Chomczynski.) Additionally, cDNA was synthesized from 1-2 μg total RNA with poly-d(T)₁₂₋₁₈ primer, obtained, for example, from Promega USA, and Moloney murine leukemia virus reverse transcriptase, obtained for example, from Promega USA.

Reverse Transcription (RT) PCR Analysis:

One μl cDNA samples were subjected to PCR amplification in 1.5 mM MgCl₂. The number of PCR cycles was 36 for all angiogenic factors but actin which was amplified using 24 cycles. For each series of primers, a positive control PCR reaction using cDNA synthesized from lung MOs mRNA extracted at time zero and a negative reaction using no template were also performed. The same primers, as described hereinabove in Table 2, were used.

Experimental Results:

Mouse Skin MOs (SMOs) implants in C57BL mice in vivo: The negative controls did not yield a detectable signal. Skin MOs transcribed all of the angiogenic factors tested (Ang1, Ang2, HGF, bFGF, three isoforms of VEGF, Ephrin 3b, and Mef2C) exhibiting expression kinetics somewhat different than that of lung MOs. In addition, angiogenic induction activity of skin MOs was at least as strong as that exhibited by lung MOs.

Comparison of angiogenesis between SMO made of old(2 years old) and young (2 months old) mice skin one month following implantation in a young mouse, revealed that no decrease in vessels formation can be detected in the old SMO (FIGS. 21A-B).

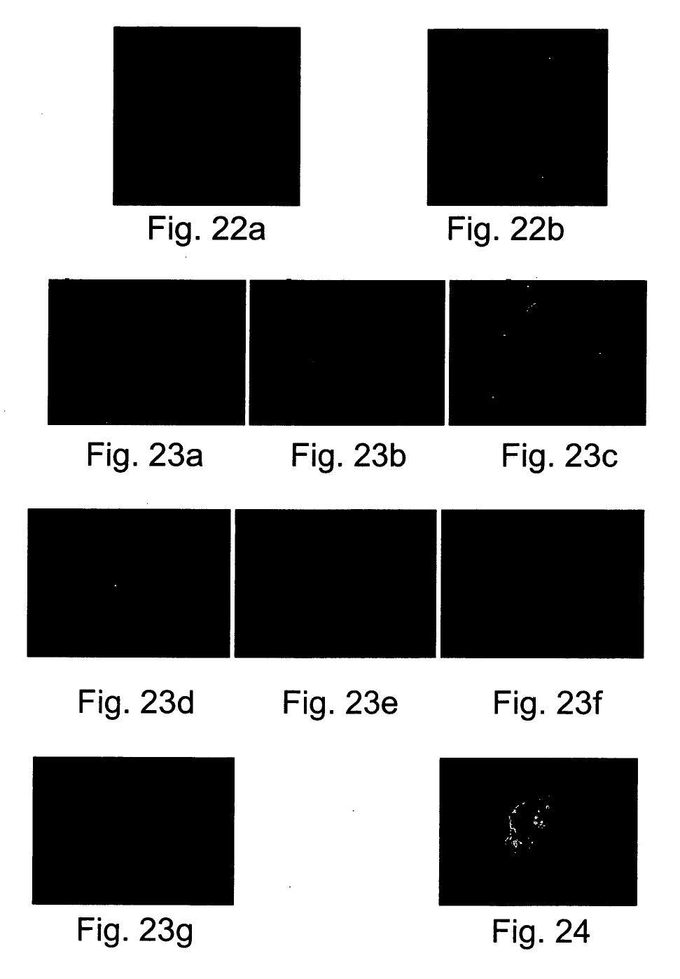

In addition, comparison of blood flow in SMOs made: of old (2 years old) and young (2 months old) mice, 2-weeks following implantation in a young mouse, revealed that blood flow in the old SMOs implanted mouse was as high as, if not higher than, that of the young SMOs implanted mouse (FIGS. 22A-B).

Thus, it is clear that SMOs made of the old mouse did not lose the capability to induce angiogenesis.

### Example 6

### Skin MOs Implantation into Rabbits

This study utilizes the methodology described hereinabove to stimulate angiogenesis in rabbits. Since a rabbit is a larger animal it can be used to more accurately model the process of angiogenesis induction in humans.

Materials and Experimental Methods:

Rabbits weighing approximately 2.5 kg each were anesthetized and a piece of skin from the center the stomach was excised and used to prepare SMOs in a manner similar to that described above for mouse SMOs. Four SMOs were implanted 5-8 mm apart in a straight line within the muscle tissue of each of the rabbit legs. Seven days following implantation, a blood flow distribution assay was performed on each rabbit using the microspheres and methodology described hereinabove.

Experimental Results:

As shown in FIGS. 23A-G, the injected microspheres were found distributed throughout the implanted SMOs indicating that blood was flowing into the SMOs and that the vascular network had further expanded throughout the whole tissue.

In addition, the average amount of beads found in unimplanted muscle tissue (FIGS. 23A and G) Was much lower than that of SMO implanted muscle tissue (FIGS. 23B-F).

Following blood flow determination, a single SMO was removed and the regional blood flow reaching directly into the SMO was determined by measuring the fluorescence intensity of the SMO. Negative control non-viable SMOs were found to yield non-detectable fluorescence and no fluorescent beads were observed inside the dead SMOs. In contrast, as shown in FIG. 24, a single viable SMO induced a significant amount of blood vessel formation as exemplified by the significant number of green fluorescent beads observed seven days following implantation into the recipient rabbit.

### Example 7

### First Device for the Preparation and Delivery of Micro-Organs

The present invention relates also to a device for the preparation and delivery of micro-organs, such as angiopumps for inducing angiogenesis in a tissue of a mammal.

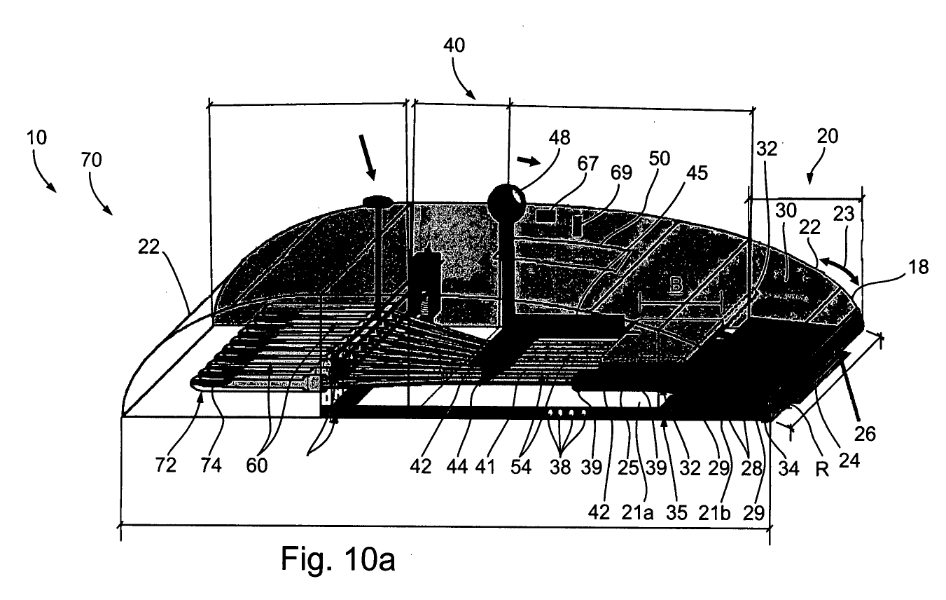

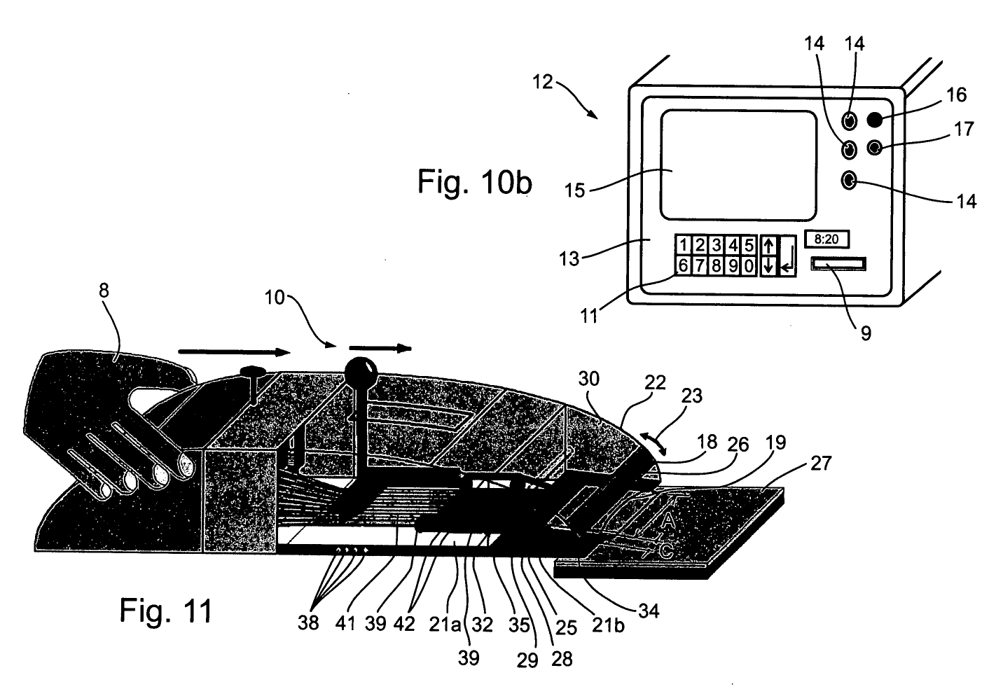

Referring further to the drawings, FIG. 10A schematically illustrates a first device 10 for micro-organ preparation and delivery, in accordance with a preferred embodiment of the present invention. First device 10 includes:

i. a tissue scraper 20, for obtaining a tissue biopsy;

ii. a tissue cutter 40, for cutting the tissue biopsy into a plurality of fragments, forming micro-organs; and

iii. at least one implanting device 60, arranged within an implanting chamber 70 and detachably coupled to first device 10, for receiving a single micro-organ, when coupled to first device 10, and for implanting the micro-organ into a subject (not shown), after decoupling from first device 10.

Additionally, first device 10 includes a casing 22, a base 21A, and a ramp 21B, which together form an enclosure that may be sealed. First device 10 is preferably about 100 mm in width and about 300 mm in length. It will be appreciated that somewhat larger or smaller dimensions are also possible.

Referring further to the drawings, FIG. 10B schematically illustrates a control system 12 for first device 10. Control system 12 may be a PC computer, a laptop, a palm computer, or the like, or a dedicated control system for first device 10, having a processor and preferably a memory. Preferably, control system 12 includes a control panel 13, having several knobs or buttons 14, a keyboard 11, a display panel 15, which may include an interactive display panel, at least one light 16, for indicating that the system is on, one or more warning lights 17, for example, to indicate that the temperature has exceeded a recommended value, or that a travel mechanism is jammed, and a read and preferably write device 9, such as a diskette drive, a CD drive, a minidisk drive, or the like, for running or recording a predetermined sequence of tasks. Additionally, control system 12 may control a plurality of devices 10 at any one tine Communication between one or more devices 10 and control system 12 may be wired or wireless.

Alternatively, no control system is used, but some functions of first device 10 are automated and controlled by knobs 38 on first device 10 (FIG. 10A). Alternatively or additionally, buttons or switches 38, or the like may be used on first device 10.

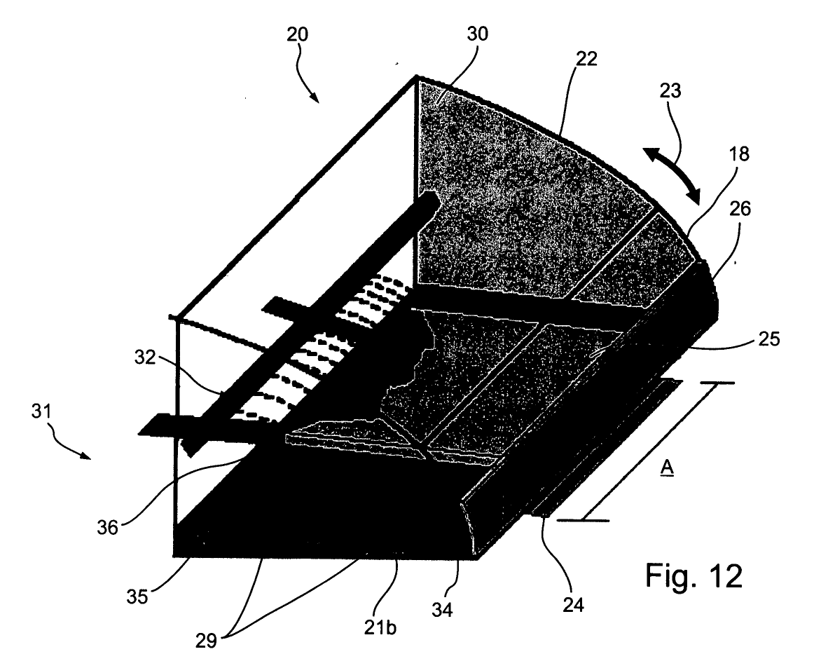

Referring further to the drawings, FIGS. 11 and 12, together with FIG. 10A, schematically illustrate tissue scraper 20, in accordance with a preferred embodiment of the present invention. Tissue scraper 20 may be for example, a standard, manually operated dermatome such as that manufactured by Robbins Instruments Inc. or by Aesculap® or a similar, preferably electrical dermatome.

Preferably, tissue scraper 20 includes a scraping blade 24 (FIG. 12), adapted for scraping a split-thickness skin biopsy (SPS) 25. A split-thickness biopsy is usually obtained by cutting, for example with a commercially available dermatome, parallel to the surface of the organ, a flat organ explant of predetermined thickness. The position of the blade determines the depth of the cutting and thus the thickness of the flat biopsy. Several illustrations of SPSs of varying thickness can be found in the literature (for example see Kondo S, Hozumi Y, Aso K., Long-term organ culture of rabbit skin: effect of EGF on epidermal structure in vitro. J Invest Dermatol. October 1990; 95(4):397-402.

Additionally, tissue scraper 20 includes casing 22 and ramp 21B. Casing 22 includes a movable portion 18, which may be raised and lowered, as shown by arrow 23. When raised, it exposes a window 19 (FIG. 11), through which SPS 25 is admitted. Additionally, movable portion 18 includes a guillotine-like blade 26, which when lowered, cuts SPS 25 off the body. The movement of movable portion 18 may be manual, or may be controlled from control system 12, or by one of switches 38 of first device 10.

Preferably, scraping blade 24 is adapted for cutting SPS 25 of a width A of preferably 6-8 mm (FIG. 11). A length B of SPS 25 is approximately 2 cm (FIG. 10A). A thickness C of SPS 25 may be about 1.0-1.4 mm, and preferably not less than about 650 microns (FIG. 11). By selecting scraping blade 24 of a predetermined width A, and by lowering guillotine-like blade 26 after a predetermined length has been scraped, both Width A and length B may be predetermined. Additionally, by adjusting the a distance R between scraping blade 24 and ramp 21B, thickness C may be predetermined. In other words, the height of the blade 24 can be lowered or raised with respect to 21B, thus affecting the thickness of the SPS. It will be appreciated that scraping blade 24 may be replaceable, for example, with a blade generating a different width A. Alternatively or additionally, blade 24 may be replaced when it grows dull.

Preferably, a region of the body 27 (FIG. 11) from which SPS 25 may be scrapped is the stomach of the patient. Alternatively, region 27 may be the back of the arm, the buttocks, the hips or another area, which is generally unexposed, and which is generally denuded of hairs. It will be appreciated that SPS 25 may be taken from another person, acting as a donor, rather then from the patient. Additionally, it will be appreciated that SPS 25 may be taken from mammals, such as primates, swines, such as wholly or partially inbred swines (e.g., miniature swines, and transgenic swines), rodents, and the like.

Prior to the scraping, region 27 is shaved, thoroughly cleaned, and disinfected using standard surgical procedures. Similarly, first device 10 is thoroughly sterilized. In a preferred embodiment first device 10 is disposed after use.

As seen in FIG. 11, the scraping operation is manual. A hand 8 of an operator pushes first device 10 into region 27 to scrape a tissue biopsy.

As seen in FIG. 12, when movable portion 18 is lowered, and guillotine-like blade 26 cuts SPS 25 off, a sealed enclosure 30 is formed around SPS 25. At least two conveyer belts 28, arranged on rollers 29 (FIG. 10A), transfer SPS 25 into sealed enclosure 30, without human contact. It will be appreciated that other automated means of transferring SPS 25 may be employed, for example, a wide conveyer belt, whose width is wider than width A of SPS 25. Alternatively, a rigid platform, seated on a moving gantry, may be used. Alternatively, other automated means of transferring SPS 25 may be employed, as known. Preferably, the transfer of transfer SPS 25 into sealed enclosure 30 is controlled from control system 12. Alternatively, it is controlled by one of switches 38 of first device 10. Alternatively, conveyer belt 28 may be manually controlled by a winding handle, or a similar mechanism.

Additionally, as seen in FIG. 12, first device 10 includes washing apparatus 31 comprising a washing-solution dispenser 32, an inlet 35 and a drain 34. Dispenser 32 sprays an appropriate washing 3 SPS 25, for thoroughly rinsing it. Washing solution 36 may be, for example, a standard culture medium DMEM with 500 units/ml Penicillin, 0.5 mg/ml Streptomycin. Washing solution 36 is admitted to dispenser 32 via inlet 35, and drains away through drain 34. Thus, rinsing takes place from the top of SPS 25. It will be appreciated that other means for rinsing SPS 25 may be employed. For example, a plurality of sprinklers may be used to spray SPS 25. Alternatively, SPS 25 may be soaked in a bath of washing solution 36, for a predetermined time. Preferably, the rinsing of SPS 25 is controlled from control system 12. Alternatively, it is controlled by one of switches 38 of first device 10. Alternatively, first device 10 is manually filled with washing solution 36, and the rinsing and drainage of washing solution 36 is powered by gravity.

As seen in FIG. 10A, following the rinsing, at least one second conveyer belt 42, and preferably two second conveyer belts 42, operating on rollers 39, transfer SPS 25 to tissue cutter 40, preferably, aseptically and preferably, without human direct intervention. It will be appreciated that other automated means of transferring SPS 25 may be employed, as was noted hereinabove. Similarly, control system 12, one of switches 38, or a manual control may be used for the automated transfer of SPS 25 to tissue cutter 40.

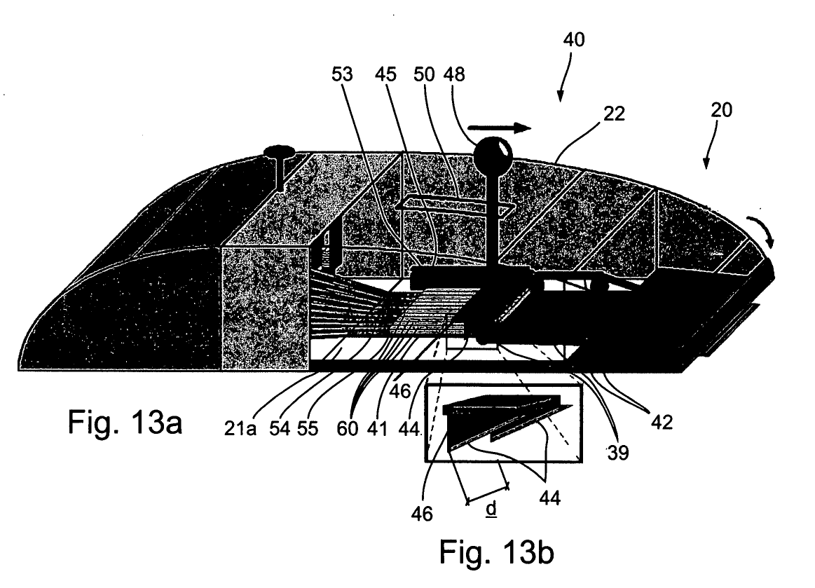

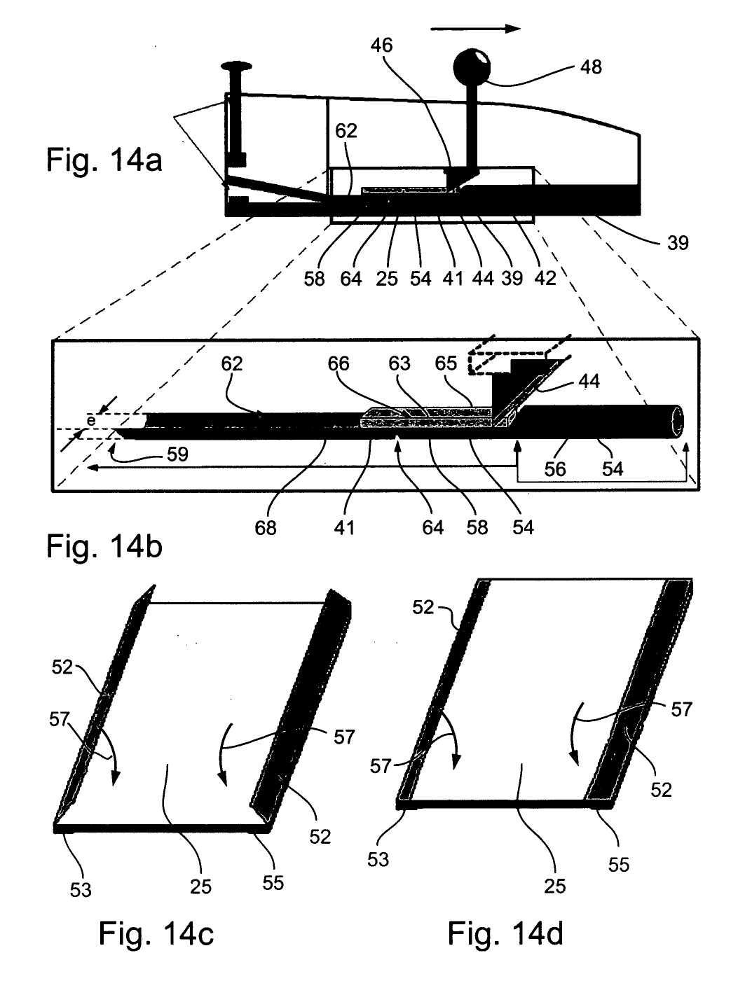

Referring further to the drawings, FIGS. 13A-13B, 14A-14D, together with FIG. 10A, schematically illustrate tissue cutter 40, in accordance with a preferred embodiment of the present invention.

As seen in FIGS. 10A and 13A, at tissue cutter 40, conveyer belts 42 transfer SPS 25 to region 41, wherein SPS 25 is supported by a plurality of rods 54, arranged in a single line, and forming micro-organ guides 54. Micro-organ guides 54 are preferably formed of medical grade polycarbonate of internal diameter approximately 0.4 mm and length approximately 16 cm. The internal diameter of micro-organ guides 54 is approximately 0.4 mm and their length is approximately 15-16 cm. It will be appreciated that somewhat smaller or larger values are also possible.

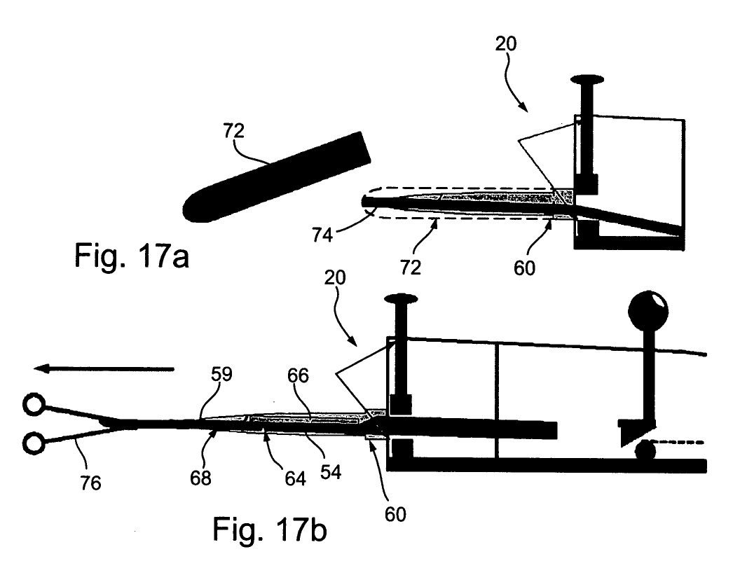

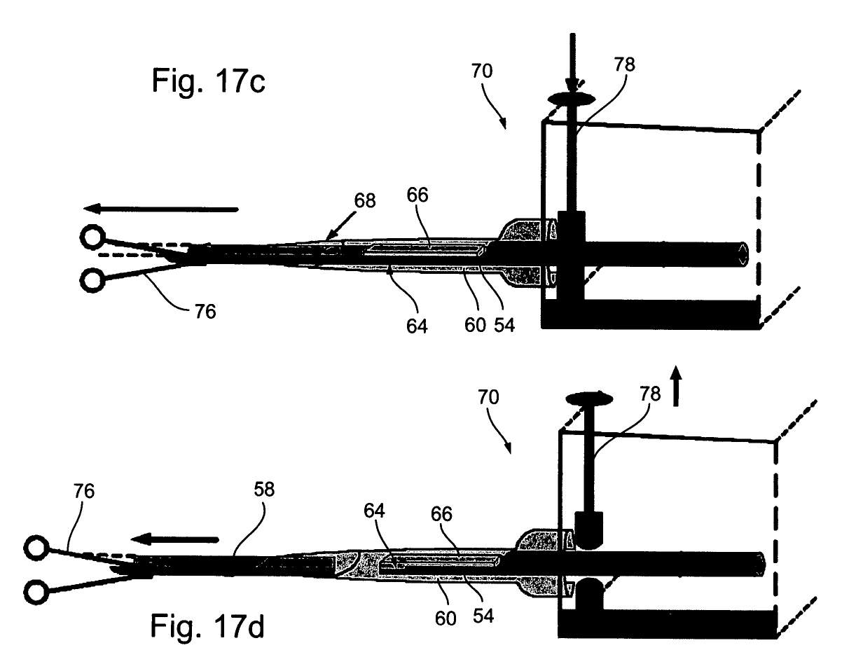

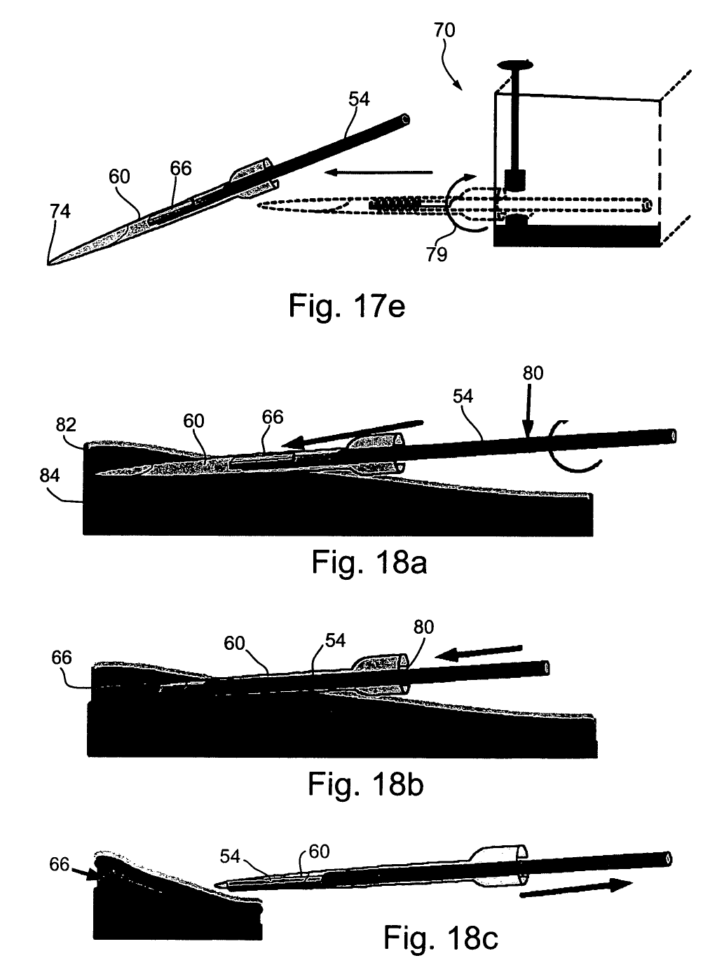

As seen in FIGS. 14A-14B, micro-organ guides 54 have a first section 56 of a circular cross section and a second section 58, which is formed as a half circle, having a concave inner surface 62. It will be appreciated that both sections 56 and 58 may be solid or hollow. However, in accordance with a preferred embodiment of the present invention, section 56 is hollow and section 58 is solid. Additionally, micro-organ guides 54 include a position marker 68, a notch 64, and a distal edge 59. The purpose of position marker 68 and notch 64 will be illustrated hereinbelow, in conjunction with FIGS. 17A-17E. Region 41, which supports SPS 25, is formed of half-split rods 58, which provide a solid flat or preferably a concave support for the SPS before being cut into micro-organs.

Additionally, as seen in FIGS. 13A and 13B, tissue cutter 40 includes a plurality of parallel, surgical-grade blades 44, arranged on a moving gantry 46, which is manually manipulated by a handle 48. Lever 48 protrudes from casing 22 through a slit window 50 which permits the manual control of gantry 46 and may further define its maximum travel. Preferably, gantry 46 glides along a straight edge 45. In accordance with an alternative embodiment, of the present invention, the travel of gantry 46 may be automated, and controlled from control system 12, or by one of switches 38 of first device 10.

In accordance with a preferred embodiment of the present invention, blades 44 are arranged at an angle with respect to SPS 25, as seen in FIGS. 13B and 14B. Alternatively, they may be rotable disc-blades, similar to rolling pizza cutters, operative to cut as they roll. Alternatively, wire cutters, similar to cheese or egg cutters, may be used. Blades 44 may have translatable angled cutting edges.

Preferably, plurality of blades 44 are adapted to operate simultaneously, as a single ensemble, and touch SPS 25 at all points at the same time, thus avoiding moving, wrinkling, or folding SPS 25 during cutting.

Blades 44 may be powered manually or by a motor. Alteratively, blades 44 may be spring loaded, and operate in a guillotine-like fashion. In accordance with an embodiment of the present invention, gantry 46 and blades 44 may be removable and replaceable, so that different types of blades 44 may be used at different times.

In accordance with the present invention, a distance d between adjacent blades 44 (FIG. 13B) is substantially equal to, or smaller than a diameter e of half rods 58, which form micro-organ guides 54 (FIG. 14B). In consequence, as seen in FIG. 14B, as blades 44 cut SPS 25 to a plurality of fragments 66, each fragment 66 (possibly, except edge fragments 53 and 55) is supported by 62 of one of half rods 58. Preferably, 11 blades are used, to cut 12 fragments 66. However, it will be appreciated that other numbers may similarily be employed.

A key feature of the present invention is distance d between adjacent blades 44. It forms the width of fragments 66. That distance is between 160 and 750 microns, and preferably 300 microns, so as to ensure that cells positioned deepest within fragment 66 are at least 80 microns and not more than about 375 microns away from a nearest surface of fragment 66. The nearest surface may be one of surfaces 63 and 65. Thus fragments 66 are operative as a micro-organ 66, or micro-organs 66.

It will be appreciated that thickness C (FIG. 11) may also be less than 750 microns (although as noted, it is more than 650 microns) thus cells positioned deepest within micro-organ 66 may be less than 375 microns away from two nearest surfaces.

In accordance with an embodiment of the present invention, gantry 46 and blades 44 may allow adjustments of distance d between adjacent blades 44, so long that distance d remains smaller than diameter e of micro-organs 54. Alternatively or additionally, gantry 46 and micro-organs 54 may be removable and replaceable, with others, of different parameters d and e.

Edge fragments 53 and 55, whose widths are generally smaller than d, are generally discarded. However, one edge fragment may be used for a viability test, as will be described hereinbelow, in conjunction with FIGS. 16A and 16B. As seen in FIG. 13B, first section 56 of micro-organ 54 preferably acts as a stop that prevents SPS 25 from sliding along concave surface 62 of one of half rods 58, by the force of blades 44.

Alternatively or additionally, as seen in FIGS. 14C and 14D, SPS 25. may be held in place, for example, by side clamps 52, or similar devices, that may close on edge fragments 53 and 55, as shown by arrows 57, to prevent wrinkling or sliding that may be brought about by the force of blades, 44. Side clamps 52 may extend the width of conveyer belts 42 (FIG. 13A), while gantry 46 and blades 44 may operate within the span of conveyer belts 42.

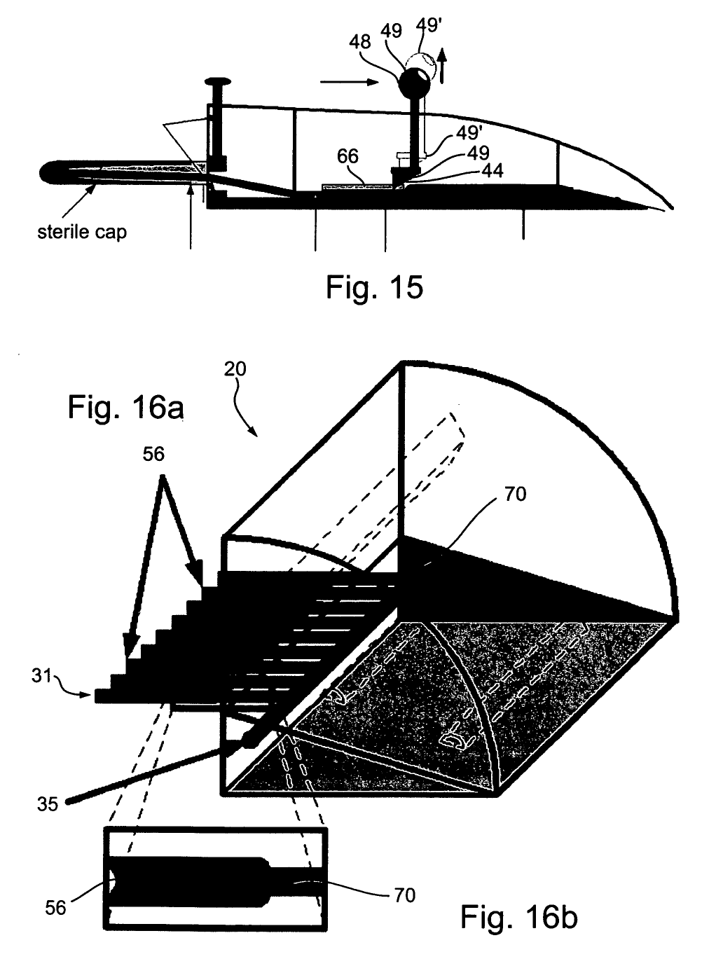

Referring further to the drawings, FIG. 15 schematically illustrates blades 44 and handle 48, when cutting is complete, in accordance with a preferred embodiment of the present invention. Preferably, when cutting is complete, blades 44 are raised from a position 49, between micro-organs 66, to a position 49′, above micro-organs 66, by raising lever 48 from its operating position 49 to locking position 49′.

It will be appreciated that first device 10 may be further operative as a sealed treatment chamber, in particular, at zone 41 (FIG. 10A). Treatment may be performed prior to cutting or after it. Treatment may include incubation at a specific temperature, wherein first device 10 may further include a heater/cooler 67 and a thermostat 69. Alternatively or additionally, treatment may include treating SPS 25 with a special solution or hormone, which may be introduced via washing apparatus 31. Treatment may be controlled from control system 12, by one of switches 38, or manually.

Referring further to the drawings, FIGS. 16A and 16B schematically illustrates applying a medium 71 for keeping micro-organs 66 moist, or for supplying nutrients, in accordance with a preferred embodiment of the present invention. Medium 71 is applied via washing apparatus 31, which may be coupled to first sections 56 of micro-organ guides 54 (FIG. 14B), which in this case are formed as hollow tubes. These lead to second sections 58, wherein micro-organs 66 are supported.

Additionally or alternatively, micro-organs 66 may be rinsed via washing apparatus 31, in a similar manner.

In accordance with the present invention, treatment may further include culturing which may require at least an hour.

Additionally or alternatively, treatment may include transformation. Transformation may comprise introducing to at least a portion of the cells of micro-organs 66 at least One exogenous polynucleotide sequence preferably selected for regulating angiogenesis. The at least one exogenous polynucleotide sequence may be integrated into a genome of the portion of the cells of micro-organs 66.

The at least one exogenous polynucleotide sequence may be designed for regulating expression of, for example, at least one angiogenic factor of a plurality of angiogenic factors. Additionally, the at least one exogenous polynucleotide sequence may include an enhancer or a suppresser sequence. Furthermore an expression product of the at least one exogenous polynucleotide sequence may be capable of regulating the expression of at least one angiogenic factor of the plurality of angiogenic factors. Additionally, the at least one exogenous polynucleotide sequence may encode at least one recombinant angiogenic factor.

Of the plurality of fragments 66, forming micro-organs 66, at least one edge fragment is discarded and another may be automatically transferred to a viability test tube, for viability testing. Viability testing can be done for example by adding MTT to the test sample. MTT is a tetrazolium salt. Dissolved MTT is converted into an insoluble purple formazan by cleavage of the terazolium ring by active mitochondrial dehydrogenase enzymes. The amount of color obtained is proportional to the viability and activity of the cells.

After rinsing and treatment, the remaining micro-organs 66 may be inserted into implanting devices 60.

Referring further to the drawings, FIGS. 17A and 17E, together with FIG. 10A, schematically illustrates the steps in inserting micro-organs 66 into implanting devices 60, in accordance with a preferred embodiment of the present invention.

As seen in FIG. 10A, a plurality of implanting devices 60 is arranged in a single line, each coupled to an micro-organ guide 54. Implanting devices 60 include slim housings 60, arranged for percutaneous insertion, protected by sterile caps 72, at their distal edges 74. They are enclosed within an implanting chamber 70, by casing 22 of first device 10.

As a first step, seen in FIG. 17A, sterile cap 72 is removed from each implanting device 60, exposing distal edge 74 of implanting device 60.

As a second step, seen in FIG. 17B, each micro-organ guide 54, on which micro-organ 66 is held, is pulled into implanting device 60, for example, by tongues 76, so that distal edge 59 of micro-organ guide 54 protrudes from implanting device 60. Micro-organ guide 54 is pulled until position marker 68 is seen at distal edge 74 f implanting device 60.

As a second step, seen in FIGS. 17C and 17D, a clamp 78, within first device 10 clamps micro-organ guide 54, while tongues 76 are used to breaks off the portion of micro-organ guide 54 distal to notch 64. The purpose of breaking off the distal portion, is to cause micro-organ 66 to be on the leading edge of micro-organ guide 54, within implanting device 60, so that leading edge will be free from guide and attach to donor tissue once partly released from implanting device 60. After the distal portion is broken off, clamp 78 releases its hold of micro-organ guide 54.

As seen in FIG. 17E, implant device 60, containing micro-organ 66 and micro-organ guide 54, is detached from implanting chamber 70 of first device 10, by rotation, as shown by arrow 79.

Referring further to the drawings, FIGS. 18A and 18C schematically illustrates the steps in implanting micro-organs 66 in a body, in accordance with a preferred embodiment of the present invention.

As a first step, shown in FIG. 18A, implanting device 60 is inserted for example but not only between a muscle 84 and a skin 82 of a body, and micro-organ guide 54 is rotated by 90 °, within implanting device 60. As a result, micro-organ 66 rests on muscle tissue 84.

As a second step, shown in FIG. 18B, micro-organ guide 54 is pushed into implanting device 60, until a position marker 80 is no longer visible, indicating that micro-organ 66 is in its implanted position.

As a third step, shown in FIG. 18C, micro-organ guide 54 is carefully pulled out, and then implanting device 60 is withdrawn.

The plurality of implanting devices 60 may be used to implant a plurality of micro-organs 66, to a subject, generally in a same area, to create a predetermined area concentration or a predetermined volume concentration of implanted micro-organs 66, in order to achieve a desired effect.

It will be appreciated that first device 10 enables preparation of micro-organs device for immediate administration, or for storage for later use, as a sterile, functional micro-organ. The description of first device 10 is given here as an example. Alternative embodiments can be envisioned that fulfill the essential features of micro-organ preparation and delivery devices.

### Example 8

### Micro-Forceps

The present invention relates a also to a micro-forceps design for the transfer and delivery of micro-organs, such as angiopumps.

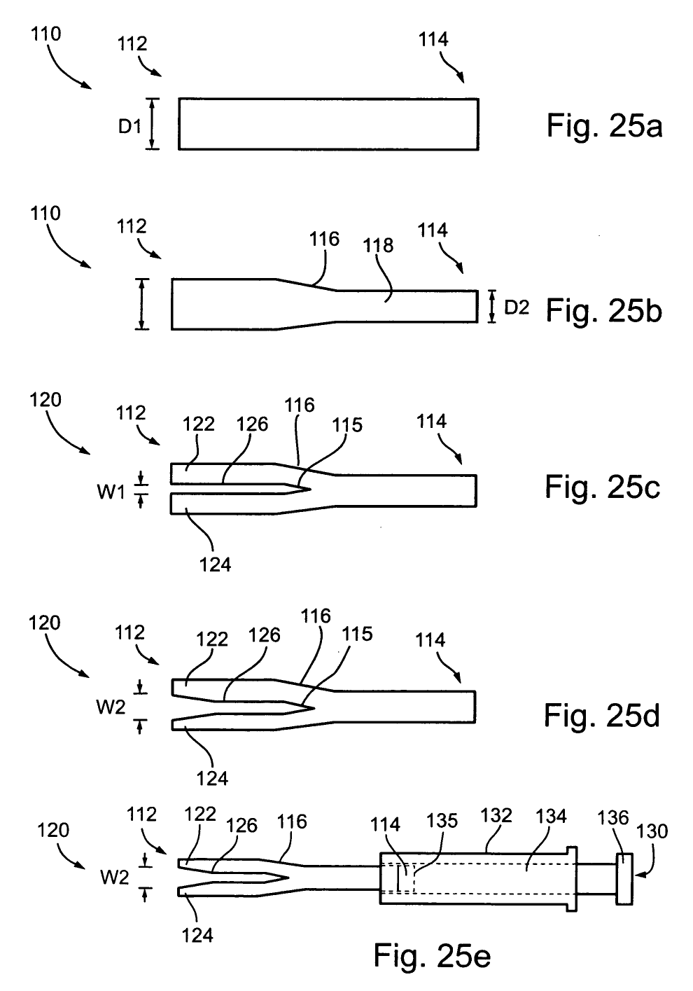

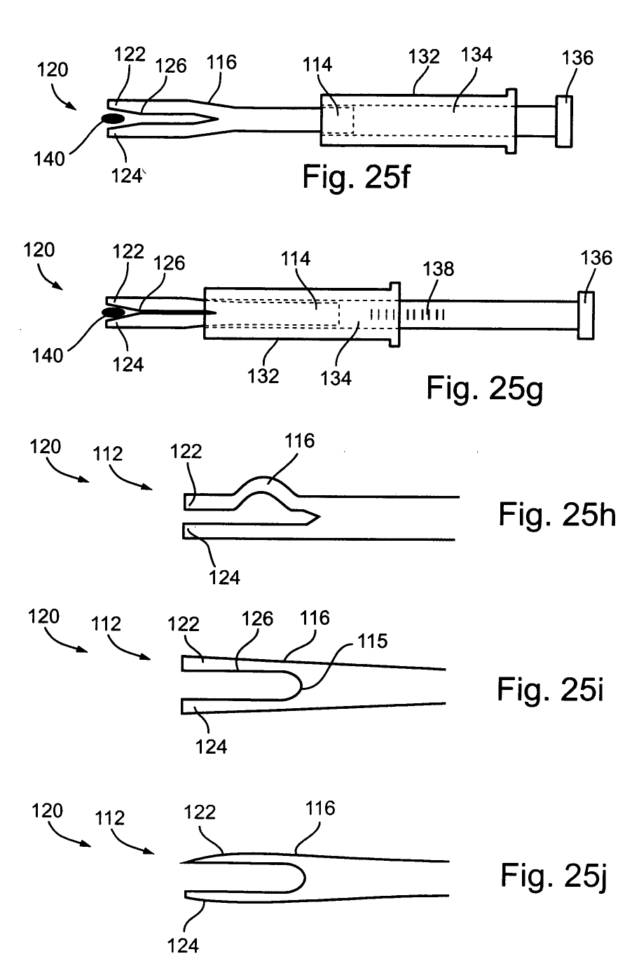

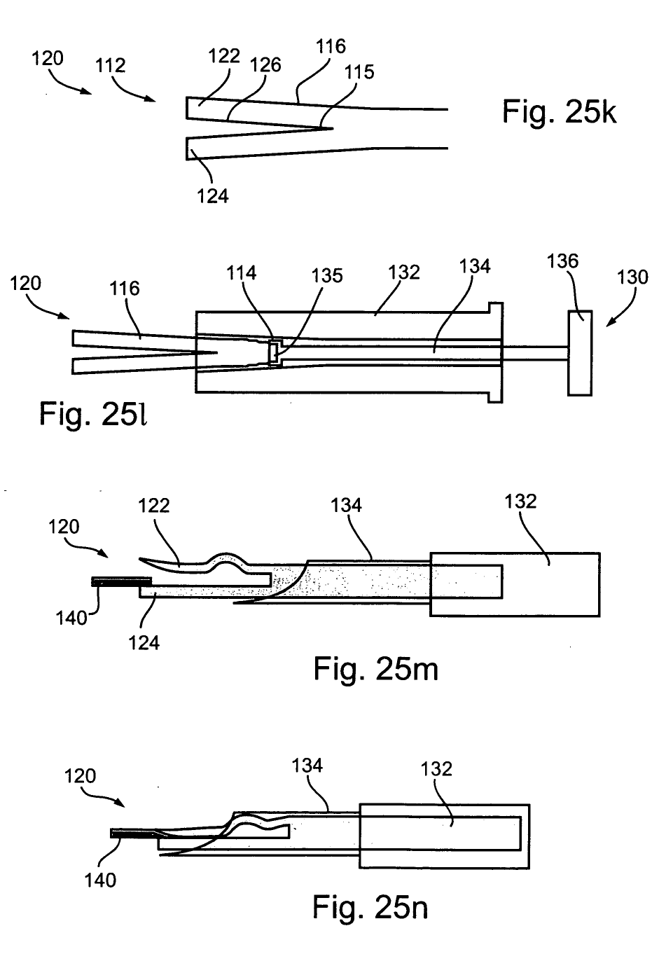

Referring further to the drawings FIGS. 25A-25N schematically illustrate stages in the fooling and operation of micro-forceps 120.

As seen in FIGS. 25A and 25B, a bar 110, having proximal and distal ends 112 and 114, respectively, with respect to the tissue, and preferably having a diameter D1 of about between 0.3 and 5 mm, is machined at distal end 114, to a somewhat lower diameter D2. Additionally, an incline 116 is produced by machining, along bar 110.

As seen in FIG. 25C, a slit 126 is made at proximal end 112, forming lips 122 and 124, with a space w1 between them, so as to create forceps 120.

Additionally, as seen in FIG. 25D, lips 122 and 124 may be filed slightly, forming a wider space w2, between them.

As seen in FIG. 25E, distal end 114 of micro-forceps 120 is inserted into a syringe 130, having a body 132 and a piston 134, wherein piston 134 includes a cap-like structure 135, adapted to rigidly fix onto distal end 114.

As seen in FIG. 25F, micro-forceps 120 may be employed to close on a tissue sample, or another sample 140.

As seen in FIG. 25G, as piston 134 is pulled distally, syringe body 132 applies a lateral force in the proximal direction, on incline 116 of forceps 120, forcing micro-forceps 120 to close and grip on target 140.

It will be appreciated that micro-forceps 120 may take other shapes, as well.

For example, as seen in FIG. 25H, Micro-forceps 120 may include a pertrusion 116, which is used to force the forceps to close.

Alternatively, as seen in FIG. 25I, micro-forceps 120 may be designed with a rounded slit 126.

As seen in FIG. 25J, the external surface of micro-forceps 120 may be filed, to make them sharper.

As seen in FIGS. 25K-25L, micro-forceps 120 may be designed with an incline, and body 132 of syringe 130 may be designed with a complimentary incline, to ensure a smooth operation.

It will be appreciated that many other designs may similarly be used, to form micro-forceps 120. Specifically, micro-forceps 120 need not be machined from a single bar. For example, a first metal bar may be used for forming one lip, and a slit may be formed in it, into which a metal strip such as a leaf spring, may be inserted, to form the second lip. The slit may be formed at an angle, to provide the slope of incline 116 (FIG. 25B). Additionally or alternatively, a screw may be used to attach the metal strip to the bar. Furthermore, a coiled spring may be used between the bar and the strip. Alternatively, two bars or two strips may be welded at an angle, to provide incline 116. It will be appreciated that many other designs are similarly possible.

FIGS. 25M and 25N illustrate a particular example, wherein micro-forceps 120 are inserted into a hyperemic needle 142, of syringe 130.

As piston 134 is pulled distally, the micro-forceps, gripping on target 140 are withdrawn into hyperemic needle 142. Hyperemic needle 142 may be further operative for implanting the micro organs.

The following is a summary of several uses of micro-forceps 120.

1. Target 140 may be a micro-organ, such as angiopumps of dimensions. 300 microns by 4 mm by 1 mm, and micro-forceps 120 enable a convenient manner of insertion of the micro organ into an injection device directly from a culture medium. In some cases, a small amount of the culture medium may also be collected by micro-forceps 120, with the target. This will allow the micro organ to remain viable within the injection device until implantation in a designated location.

2. Target 140 may be a mammalian oocyte for in vitro fertilization, held firmly by micro-forceps 140 for sperm injection or nuclear transfer into the oocyte.

3. Target 140 may be a clump or cellular aggregate made of stem cells that may be genetically modified for insertion into a pre-defined position in the body through a catheter and or surgical needle.

4. Target 140 may be a non-biologic object, such as a micro circuit.

### Example 9

### Second Device for the Preparation and Delivery of Micro-Organs

The present invention relates also to a second device 100 for the preparation and delivery of micro-organs, such as angiopumps, for inducing angiogenesis in a tissue of a mammal.

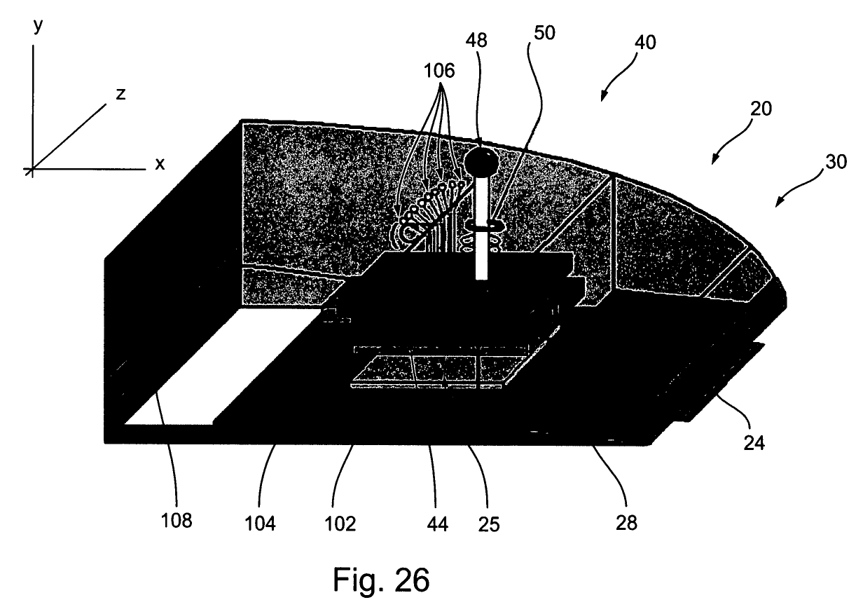

Referring further to the drawing's, FIG. 26 schematically illustrates second device 100 for micro-organ preparation and delivery in accordance with a preferred embodiment of the present invention. Second device 100 defines an x;y;z coordinate system, and includes tissue scraper 20, which is substantially as described hereinabove, in conjunction with first device 10 of FIGS. 10A, 11, and 12. Tissue scraper 20 includes sealed enclosure 30, scraping blade 24, adapted for scraping split-thickness skin biopsy (SPS) 25, and conveyer belts 28, for the transfer of SPS 25 to sealed enclosure 30.

However, tissue cutter 40, for cutting the tissue biopsy into a plurality of fragments 66 (FIG. 14B), to form the micro-organs, and the mode of transfer of the micro-organs into the implanting devices is different than that of device 10 (FIG. 10B).

In second device 100, tissue cutter 40 includes a baseboard 102, on which SPS 25 is placed by conveyer belts 28. Additionally, plurality of parallel, surgical-grade blades 44 are adapted for motion in the ±y direction, so as to cut SPS 25 into thin slices. As before, the distance between adjacent blades 44 is between 160 and 750 microns, and preferably 300 microns, to ensure that cells positioned deepest within the micro-organs (fragment 66 of FIG. 14B) are at least about 80 microns and not more than about 375 microns away from a nearest surface of the micro-organ.

Plurality of blades 44 is preferably manually manipulated by handle 48, which protrudes from casing 22 through round window 50. Preferably, lever 48 is spring loaded, operating in a guillotine-like fashion, to release plurality of blades 44 simultaneously, as a single ensemble, so that blades 44 touch SPS 25 at all points, at the same time, avoiding moving, wrinkling, or folding SPS 25 during cutting. Alternatively, lever 48 may be powered by a motor.

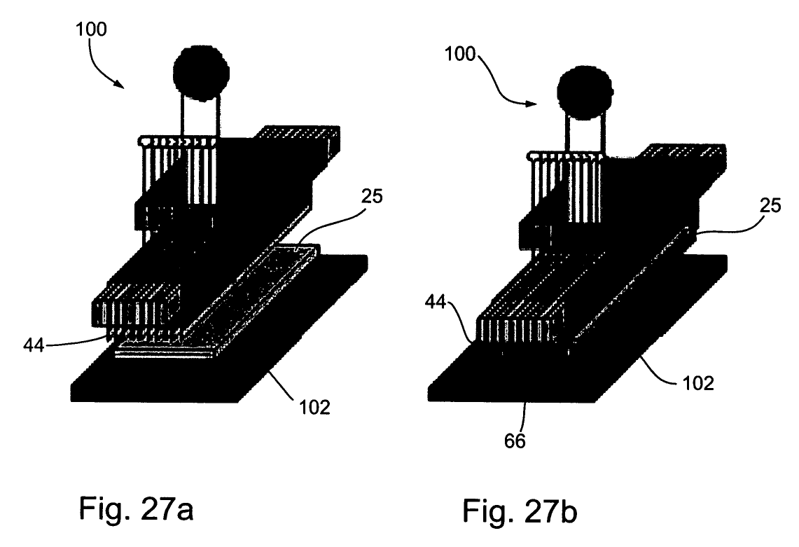

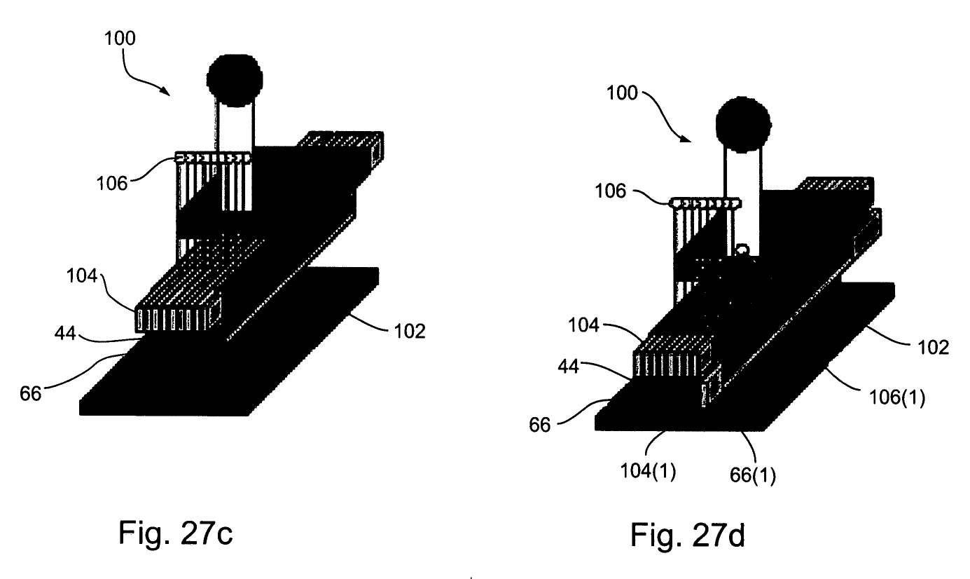

Referring further to the drawings, FIGS. 27A-27D schematically illustrate second device 100 with the cutting stages of SPS 25 by plurality of blades 44, in accordance with a preferred embodiment of the present invention.

As seen in FIG. 27A, SPS 25 is placed on baseboard 102, with plurality of blades 44 above it.

As seen in FIG. 27B, plurality of blades 44 is dropped, forming a plurality of fragments 66, between them, on baseboard 102.

As seen in FIG. 27C, plurality of blades 44, is lifted manually, and fragments 66 are lifted with blades 44, by frictional forces. A dedicated plurality of plungers 104, operative by dedicated levers 106, are used to push fragments 66 out from between blades 44, onto baseboard 102. Dedicated plungers 104 are used one at a time.

As seen in FIG. 27D, one of dedicated plunger 104, namely, a dedicated plunger 104(1) is manually released by a dedicated lever 106(1), pushing a fragment 66(1) from between blades 44 and onto baseboard 102.

Referring further to the drawings, FIGS. 28A-28C, 29A-29C, and FIG. 26, together, schematically illustrate the use of micro-forceps 120 in the loading stages of fragment 66(1), which was lowered onto baseboard 102 in FIG. 27D, hereinabove.

As seen in FIG. 26, device 100 includes a hole 108, adapted to slide in the ±z direction, for example, by using two plates that may slide against each other. Hole 108 is provided so as to minimize the opening and keep fragments 66 generally in a sealed environment.

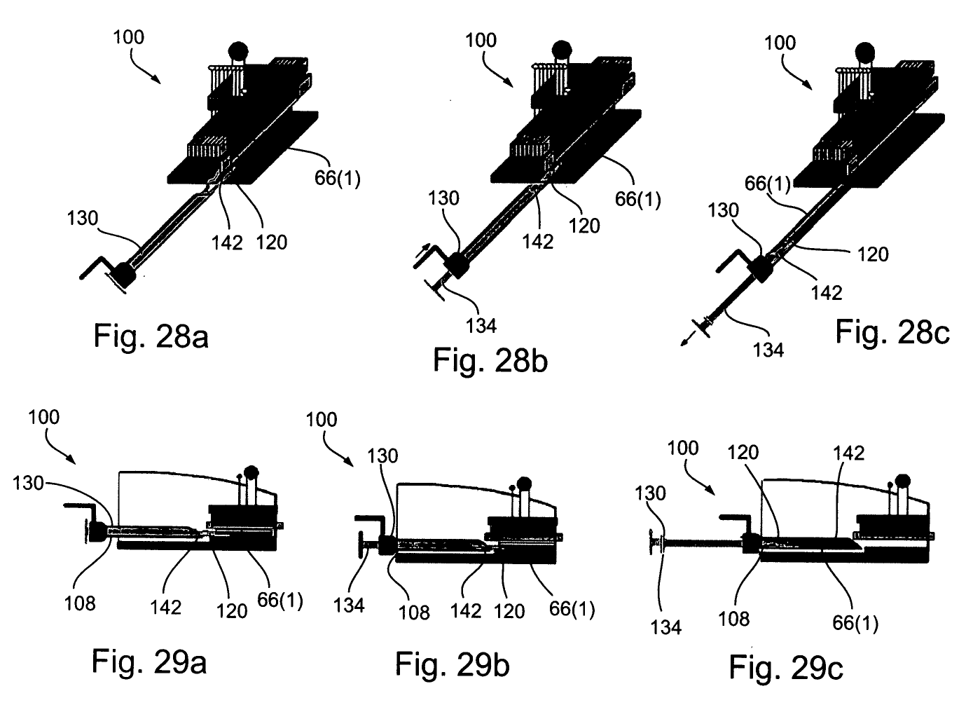

As seen in FIGS. 28A and 29A, micro forceps 120, arranged on syringe 130, preferably, within hyperemic needle 142 (FIGS. 15M-25N), may be inserted through hole 108 of device 100, when hole 108 is aligned with fragment 66(1) on baseboard 102.

As seen in FIGS. 28B and 29B, micro forceps 120 grip fragment 66(1).

As seen in FIGS. 28C and 29C, piston 134 is withdrawn, so as to draw fragment 66(1) into hyperemic needle 142.

Hyperemic needle 142 may be used for implanting fragment 66(1), directly.

It is appreciated that certain features of the invention, which are, for clarity, described in the context of separate embodiments, may also be provided in combination in a single embodiment. Conversely, various features of the invention, which are for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-173. (canceled)

174. An apparatus for generating micro-organs from a tissue biopsy and for administering the micro-organs into a subject, the apparatus comprising:
(a) a cutting chamber for cutting the tissue biopsy into a plurality of micro-organs; and
(b) an implanting mechanism for administering the plurality of micro-organs into the subject, said implanting mechanism being operably coupled to said cutting chamber.

175. The apparatus of claim 174, further comprising a viability testing chamber operably coupled to said cutting chamber for testing a viability of at least one sacrificial micro-organ of said plurality of micro-organs.

176. The apparatus of claim 174, wherein said implanting mechanism comprises a multi-channel implanter and corresponding advancing elements for advancing said plurality of micro-organs from said cutting chamber to said multi-channel implanter and further for administering the plurality of micro-organs into the subject.

177. The apparatus of claim 174, further comprising a processing chamber being operably coupled to said cutting chamber and said implanting mechanism for processing said micro-organs prior to said administering.

178. The apparatus of claim 174, wherein said cutting chamber is designed and constructed such that once the tissue biopsy is cut into said plurality of micro-organs, each of said micro-organs such that cells positioned deepest within a micro-organ of said plurality of micro-organs are at least about 80-100 microns and not more than 225-375 microns away from a nearest surface of said micro-organ.

179. The apparatus of claim 174, wherein said cutting chamber comprises a cutting mechanism having a plurality of blades movable to cut the tissue biopsy into said plurality of micro-organs.

180. The apparatus of claim 179, wherein said blades are so disposed with respect to one another such that once the tissue biopsy is cut into said plurality of micro-organs, each of said micro-organs such that cells positioned deepest within a micro-organ of said plurality of micro-organs are at least about 80-100 microns and not more than 225-375 microns away from a nearest surface of said micro-organ.

181. The apparatus of claim 174, wherein said implanting mechanism comprises a syringe-operated micro-forceps, inserted within a hyperemic needle of said syringe, said-hyperemic needle being operative for administering the micro-organs into the subject.

182. The apparatus of claim 181, wherein said syringe-operated micro-forceps is further operative for removing the micro-organs from said apparatus and into said hyperemic needle.

183. An apparatus for generating micro-organs from a tissue biopsy, the apparatus comprising:
(a) a cutting chamber for cutting the tissue biopsy into a plurality of micro-organs; and
(b) a viability testing chamber operably coupled to said cutting chamber for testing a viability of at least one sacrificial micro-organ of said plurality of micro-organs.

184. The apparatus of claim 183, wherein each of said plurality of blades has a translatable angled cutting edge.

185. The apparatus of claim 183, wherein each of said plurality of blades is a rotatable disc-blade.

186. An apparatus for generating micro-organs from a tissue biopsy, the apparatus comprising:
(a) a cutting chamber for cutting the tissue biopsy into a plurality of micro-organs;
(b) a processing chamber being operably coupled to said cutting chamber for processing said micro-organs; and
(c) an advancing mechanism for advancing said micro-organs from said cutting chamber into said processing chamber.

187. A method of generating micro-organs from a tissue biopsy comprising:
providing an apparatus which comprises:
(a) a cutting chamber for cutting the tissue biopsy into a plurality of micro-organs; and
(b) an implanting mechanism for administering the plurality of micro-organs into the subject, said implanting mechanism being operably coupled to said cutting chamber; and
placing the tissue biopsy is said cutting chamber and cutting the tissue biopsy into the plurality of micro-organs.

188. The method of claim 187, wherein the micro-organs are operable as angiopumps.

189. The method of claim 187, wherein said apparatus further comprises a viability testing chamber operably coupled to said cutting chamber for testing a viability of at least one sacrificial micro-organ of said plurality of micro-organs, the method further comprising testing said viability of said at least one sacrificial micro-organ of said plurality of micro-organs prior to using said implanting mechanism for administering the plurality of micro-organs into the subject.

190. A device for micro-organ preparation and delivery, comprising:
a tissue cutter, for cutting a tissue biopsy into a plurality of fragments, forming a plurality of micro-organs: and
at least one implanting device, detachably coupled to said tissue cutter, for receiving a micro-organ, of said plurality of micro-organs, when coupled to said tissue cutter, and for implanting said micro-organ into a subject, after decoupling from said tissue cutter.

191. The device of claim 190, further comprising a tissue scraper, for obtaining said tissue biopsy.

192. The device of claim 191, wherein said tissue scraper is adapted for preparing said tissue biopsy to a predetermined parameter, selected from the group consisting of a width, a length, and a thickness.

193. The device of claim 191, wherein said tissue scraper has a replaceable blade.

194. The device of claim 190, wherein said device is sealed within a base, a ramp, and a casing.

195. The device of claim 190, wherein said device includes a control system.

196. The device of claim 190, wherein said device includes at least one automated travel mechanism for transferring the tissue biopsy from one region of said device to another.

197. The device of claim 190, wherein said device includes a washing apparatus for rinsing the tissue biopsy.

198. The device of claim 197, wherein said washing apparatus is further operative for applying a medium to the tissue biopsy.

199. The device of claim 190, wherein said device is further operative as a tissue treatment chamber.

200. The device of claim 190, wherein said tissue cutter comprises a plurality of parallel, surgical-grade blades, designed to cut the tissue biopsy into said plurality of fragments, forming said micro-organs, such that cells positioned deepest within any one of said micro-organs are at least about 80-100 microns and not more than about 225-375 microns away from a nearest surface.

201. The device of claim 190, wherein said tissue cutter comprises a plurality of parallel surgical-grade blades, arranged at an angle to the tissue biopsy.

202. The device of claim 190, wherein said tissue cutter comprises a plurality of parallel surgical-grade blades, arranged as rotatable disc-blades.

203. The device of claim 190, wherein said device comprises a viability testing chamber for testing a viability of at least one micro-organ of said plurality of micro-organs.

204. The device of claim 190, wherein said tissue cutter is operative to cut the tissue biopsy, to form said micro-organs, and to arrange each of said micro-organs on a single guide of a plurality of guides, in a single operation.

205. The device of claim 203, wherein said at least one implanting device includes a slim housing, adapted for percutaneous insertion, and operable to receive one of said plurality of guides.

206. The device of claim 203, wherein said at least one implanting device includes a plurality of implanting devices, each operable to receive one of said plurality of guides.

207. A method for micro-organ preparation, comprising:
employing a device for micro-organ preparation and delivery, which includes:
a tissue scraper, for obtaining a tissue biopsy;
a tissue cutter, for cutting the tissue biopsy into a plurality of fragments, forming a plurality of micro-organs: and
at least one implanting device, detachably coupled to said tissue cutter, for receiving a micro-organ, of said plurality of micro-organs, when coupled to said tissue cutter, and for implanting said micro-organ into a subject, after decoupling from said tissue cutter;
scraping the tissue biopsy, with said tissue scraper;
cutting the tissue biopsy to said plurality of fragments, forming said plurality of micro-organs, with said tissue cutter;
mounting said micro-organ, of said plurality of micro-organs, on said at least one implanting device; and
decoupling said at least one implanting device.

208. The method of claim 207, wherein said device includes at least one automated travel mechanism for transferring the tissue biopsy from one region of said device to another.

209. The method of claim 207, wherein the tissue biopsy is derived from a tissue or organ selected from the group consisting of lung, liver, kidney, muscle, spleen, skin, heart, lymph node and bone marrow.

210. The method of claim 207, wherein said cutting includes cutting the tissue biopsy into said plurality of fragments, forming said micro-organs, such that cells positioned deepest within any one of said micro-organs are at least about 80 microns and not more than about 375 microns away from a nearest surface.

211. The method of claim 207, wherein said cutting includes cutting the tissue biopsy into said plurality of fragments, forming said micro-organs, such that cells positioned deepest within any one of said micro-organs are at least about 100 microns and not more than about 225 microns away from a nearest surface.

212. The method of claim 207, wherein said tissue biopsy is a split-thickness tissue biopsy.

213. A micro-forceps comprising:
an elongated body, which defines an overall cross-sectional diameter of between 0.3 and 5 mm and proximal and distal ends, with respect to a target, said elongated body comprising:
two lips, at said proximal end, defining a clearance between them; and
a diametric increase, in the overall cross-sectional diameter, along said elongated body, adapted to force said two lips against each other, when a lateral force in the proximal direction is applied to said diametric increase.

214. The micro-forceps of claim 213, wherein said diametric increase is a fold along at least one of said lips.

215. The micro-forceps of claim 213, wherein said diametric increase is an incline along at least one of said lips.

216. The micro-forceps of claim 213, adapted for operation with a syringe, into which said elongated body may be inserted, wherein said syringe has an internal diameter that is smaller than said diametric increase, said syringe further having a piston fixedly attached to said distal end of said elongated body, so that as said piston is drawn into said syringe, a lateral force in the proximal direction is applied to said diametric increase, by said syringe, forcing said lips to close and grip said target.

217. The micro-forceps of claim 216 and further including a hyperemic needle, into which said elongated body may be inserted, wherein said hyperemic needle has an internal diameter that is smaller than said diametric increase, and wherein said hyperemic needle, manipulated by said syringe, is operative for applying said lateral force in the proximal direction, to said diametric increase, forcing said lips to close and grip said target.

218. The micro-forceps of claim 213, adapted for operation with a catheter, into which said elongated body may be inserted, wherein said catheter has an internal diameter that is smaller than said diametric increase, and wherein said catheter applies said lateral force in the proximal direction to said diametric increase, forcing said lips to close and grip said target.


SEQUENCE LISTING (8 sequences)

SEQ ID NO: 1 - DNA, 21 bp, Artificial sequence
  Single strand DNA oligonucleotide
cgtgggtggaggagggtggac

SEQ ID NO: 2 - DNA, 21 bp, Artificial sequence
  Single strand DNA oligonucleotide
tgcgtcaaaccaccagcctcc

SEQ ID NO: 3 - DNA, 20 bp, Artificial sequence
  Single strand DNA oligonucleotide
taccacaggcattgtgatgg

SEQ ID NO: 4 - DNA, 20 bp, Artificial sequence
  Single strand DNA oligonucleotide
aatagtgatgacctggccgt

SEQ ID NO: 5 - DNA, 20 bp, Artificial sequence
  Single strand DNA oligonucleotide
ggtcacacagggacagcagg

SEQ ID NO: 6 - DNA, 21 bp, Artificial sequence
  Single strand DNA oligonucleotide
ccaagggccggatcagcatgg

SEQ ID NO: 7 - DNA, 19 bp, Artificial sequence
  Single strand DNA oligonucleotide
actttctgctctcttgggt

SEQ ID NO: 8 - DNA, 18 bp, Artificial sequence
  Single strand DNA oligonucleotide
ccgccttggcttgtcaca